SEQUENCE LISTING (697 sequences)

SEQ ID NO: 1 - protein, 12 aa, Homo sapiens
LWVNPSEFLPER

SEQ ID NO: 2 - protein, 14 aa, Homo sapiens
QLDENANVQLSDEK

SEQ ID NO: 3 - protein, 11 aa, Homo sapiens
YLPNPSLNAFK

SEQ ID NO: 4 - protein, 10 aa, Homo sapiens
FLTPDGAIDK

SEQ ID NO: 5 - protein, 8 aa, Homo sapiens
ASRPQVPK

SEQ ID NO: 6 - protein, 11 aa, Homo sapiens
TVQEHYQDFDK

SEQ ID NO: 7 - protein, 9 aa, Homo sapiens
YLPNPALQR

SEQ ID NO: 8 - protein, 7 aa, Homo sapiens
FLWFLQK

SEQ ID NO: 9 - protein, 7 aa, Homo sapiens
YPDVQTR

SEQ ID NO: 10 - protein, 12 aa, Homo sapiens
QVLEGHVLSEAR

SEQ ID NO: 11 - protein, 8 aa, Homo sapiens
ELVALLVR

SEQ ID NO: 12 - protein, 9 aa, Homo sapiens
YSHDDPEFR

SEQ ID NO: 13 - protein, 8 aa, Homo sapiens
ANPNEPAK

SEQ ID NO: 14 - protein, 11 aa, Homo sapiens
ELLSHNEEFGR

SEQ ID NO: 15 - protein, 15 aa, Homo sapiens
GTGGANIDPTFFLSR

SEQ ID NO: 16 - protein, 11 aa, Homo sapiens
GYGVVFSNGER

SEQ ID NO: 17 - protein, 13 aa, Homo sapiens
IQEEAGFLIDALR

SEQ ID NO: 18 - protein, 13 aa, Homo sapiens
IQEESGFLIEAIR

SEQ ID NO: 19 - protein, 7 aa, Homo sapiens
FAIATLR

SEQ ID NO: 20 - protein, 15 aa, Homo sapiens
GTHGANIDPTFFLSR

SEQ ID NO: 21 - protein, 11 aa, Homo sapiens
GEQATFDWLFK

SEQ ID NO: 22 - protein, 7 aa, Homo sapiens
AEAFSGR

SEQ ID NO: 23 - protein, 7 aa, Homo sapiens
YPHVAER

SEQ ID NO: 24 - protein, 7 aa, Homo sapiens
YFPGAHR

SEQ ID NO: 25 - protein, 11 aa, Homo sapiens
GYGVIFANGNR

SEQ ID NO: 26 - protein, 11 aa, Homo sapiens
VQEEIDHVIGR

SEQ ID NO: 27 - protein, 12 aa, Homo sapiens
EHQASLDVNNPR

SEQ ID NO: 28 - protein, 8 aa, Homo sapiens
GNSPISQR

SEQ ID NO: 29 - protein, 13 aa, Homo sapiens
EALIDNGEEFSGR

SEQ ID NO: 30 - protein, 8 aa, Homo sapiens
GIFPLAER

SEQ ID NO: 31 - protein, 16 aa, Homo sapiens
GTTILISLTSVLHDNK

SEQ ID NO: 32 - protein, 9 aa, Homo sapiens
IAENFAYIK

SEQ ID NO: 33 - protein, 13 aa, Homo sapiens
EALIDHGEEFSGR

SEQ ID NO: 34 - protein, 7 aa, Homo sapiens
YPEVTAK

SEQ ID NO: 35 - protein, 8 aa, Homo sapiens
GHFPLAER

SEQ ID NO: 36 - protein, 14 aa, Homo sapiens
IYGPVFTLYFGLER

SEQ ID NO: 37 - protein, 16 aa, Homo sapiens
GTTILTSLTSVLHDNK

SEQ ID NO: 38 - protein, 8 aa, Homo sapiens
SQGVFLAR

SEQ ID NO: 39 - protein, 8 aa, Homo sapiens
DIEVQGFR

SEQ ID NO: 40 - protein, 15 aa, Homo sapiens
FITLVPSNLPHEATR

SEQ ID NO: 41 - protein, 7 aa, Homo sapiens
EALLDYK

SEQ ID NO: 42 - protein, 7 aa, Homo sapiens
YPEIEEK

SEQ ID NO: 43 - protein, 10 aa, Homo sapiens
GDLPAFHAHR

SEQ ID NO: 44 - protein, 11 aa, Homo sapiens
GIIFNNGPTWK

SEQ ID NO: 45 - protein, 12 aa, Homo sapiens
FKPEHFLNENGK

SEQ ID NO: 46 - protein, 7 aa, Homo sapiens
LHEEIDR

SEQ ID NO: 47 - protein, 11 aa, Homo sapiens
VQEEIDLVVGR

SEQ ID NO: 48 - protein, 13 aa, Homo sapiens
VIGQGQQPSTAAR

SEQ ID NO: 49 - protein, 8 aa, Homo sapiens
DFIDAYLK

SEQ ID NO: 50 - protein, 15 aa, Homo sapiens
EVTVDTTLAGYHLPK

SEQ ID NO: 51 - protein, 13 aa, Homo sapiens
IQEEAQHLTEAIK

SEQ ID NO: 52 - protein, 12 aa, Homo sapiens
EENGQPFDPHFK

SEQ ID NO: 53 - protein, 12 aa, Homo sapiens
LLDEVTYLEASK

SEQ ID NO: 54 - protein, 10 aa, Homo sapiens
FTFRPPNNEK

SEQ ID NO: 55 - protein, 13 aa, Homo sapiens
FEYQDSWFQQLLK

SEQ ID NO: 56 - protein, 16 aa, Homo sapiens
FHLHFPHELVPDLKPR

SEQ ID NO: 57 - protein, 7 aa, Homo sapiens
GRPFDFK

SEQ ID NO: 58 - protein, 9 aa, Homo sapiens
DPEVFHPER

SEQ ID NO: 59 - protein, 16 aa, Homo sapiens
GTTVITNLYSVHFDEK

SEQ ID NO: 60 - protein, 7 aa, Homo sapiens
YFGYGQK

SEQ ID NO: 61 - protein, 9 aa, Homo sapiens
NDPSSTFSK

SEQ ID NO: 62 - protein, 10 aa, Homo sapiens
FFNDAIETYK

SEQ ID NO: 63 - protein, 11 aa, Homo sapiens
NAAVVYDFLSR

SEQ ID NO: 64 - protein, 7 aa, Homo sapiens
EFQAVVR

SEQ ID NO: 65 - protein, 9 aa, Homo sapiens
DLVDAFLLK

SEQ ID NO: 66 - protein, 9 aa, Homo sapiens
HPEEFNPDR

SEQ ID NO: 67 - protein, 13 aa, Homo sapiens
EALGGQAEEFSGR

SEQ ID NO: 68 - protein, 13 aa, Homo sapiens
ELGAGQAPSLGDR

SEQ ID NO: 69 - protein, 15 aa, Homo sapiens
QLLHHVSTLAAFTVR

SEQ ID NO: 70 - protein, 7 aa, Homo sapiens
FLDADGR

SEQ ID NO: 71 - protein, 16 aa, Homo sapiens
QVQQHQGNLDASGPAR

SEQ ID NO: 72 - protein, 13 aa, Homo sapiens
LPYTDAVLHEAQR

SEQ ID NO: 73 - protein, 9 aa, Homo sapiens
ALHSLGVGR

SEQ ID NO: 74 - protein, 11 aa, Homo sapiens
GGGIFFSSGAR

SEQ ID NO: 75 - protein, 10 aa, Homo sapiens
FITLLPHVPR

SEQ ID NO: 76 - protein, 11 aa, Homo sapiens
TVVLTGFEAVK

SEQ ID NO: 77 - protein, 7 aa, Homo sapiens
HPDVQGR

SEQ ID NO: 78 - protein, 13 aa, Homo sapiens
TELFLLFAGLLQR

SEQ ID NO: 79 - protein, 11 aa, Homo sapiens
YGPVFTVHLGR

SEQ ID NO: 80 - protein, 11 aa, Homo sapiens
SLLSPTFTSGK

SEQ ID NO: 81 - protein, 12 aa, Homo sapiens
LQEEIDAVLPNK

SEQ ID NO: 82 - protein, 7 aa, Homo sapiens
DGTLSGE

SEQ ID NO: 83 - protein, 8 aa, Homo sapiens
DTINFLSK

SEQ ID NO: 84 - protein, 12 aa, Homo sapiens
YWTEPEEFRPER

SEQ ID NO: 85 - protein, 12 aa, Homo sapiens
FNPLDPFVLSIK

SEQ ID NO: 86 - protein, 7 aa, Homo sapiens
DETVSGA

SEQ ID NO: 87 - protein, 9 aa, Homo sapiens
EIDTVLPNK

SEQ ID NO: 88 - protein, 14 aa, Homo sapiens
FGGLLLTEKPIVLK

SEQ ID NO: 89 - protein, 7 aa, Homo sapiens
LFPVVSR

SEQ ID NO: 90 - protein, 11 aa, Homo sapiens
TLLSPAFTSVK

SEQ ID NO: 91 - protein, 9 aa, Homo sapiens
YIPFGAGPR

SEQ ID NO: 92 - protein, 7 aa, Homo sapiens
DSIDLYR

SEQ ID NO: 93 - protein, 7 aa, Homo sapiens
GLWNFDR

SEQ ID NO: 94 - protein, 7 aa, Homo sapiens
QEAENSK

SEQ ID NO: 95 - protein, 7 aa, Homo sapiens
FALTNIK

SEQ ID NO: 96 - protein, 16 aa, Homo sapiens
LDNLPILQPEKPIVLK

SEQ ID NO: 97 - protein, 8 aa, Homo sapiens
AVQLYLHR

SEQ ID NO: 98 - protein, 9 aa, Homo sapiens
VATALTLLR

SEQ ID NO: 99 - protein, 9 aa, Homo sapiens
FELLPDPTR

SEQ ID NO: 100 - protein, 7 aa, Homo sapiens
IPIPIAR

SEQ ID NO: 101 - protein, 12 aa, Homo sapiens
ELSTPVTFPDGR

SEQ ID NO: 102 - protein, 16 aa, Homo sapiens
NGFHLHLKPLGPGSGK

SEQ ID NO: 103 - protein, 16 aa, Homo sapiens
EILGDQDFFQWDDLGK

SEQ ID NO: 104 - protein, 10 aa, Homo sapiens
LYPPVPQVYR

SEQ ID NO: 105 - protein, 15 aa, Homo sapiens
NSAVWPDPEVFDSLR

SEQ ID NO: 106 - protein, 8 aa, Homo sapiens
GDTGLGHR

SEQ ID NO: 107 - protein, 9 aa, Homo sapiens
FEFSLDPSR

SEQ ID NO: 108 - protein, 7 aa, Homo sapiens
LSDADLR

SEQ ID NO: 109 - protein, 9 aa, Homo sapiens
GLLVLEGPK

SEQ ID NO: 110 - protein, 14 aa, Homo sapiens
APDVYDFFLQWIGR

SEQ ID NO: 111 - protein, 12 aa, Homo sapiens
QLSKPVTFVDGR

SEQ ID NO: 112 - protein, 7 aa, Homo sapiens
FDPENIK

SEQ ID NO: 113 - protein, 12 aa, Homo sapiens
HVTQDIVLPDGR

SEQ ID NO: 114 - protein, 14 aa, Homo sapiens
VLTQLVATYPQGFK

SEQ ID NO: 115 - protein, 10 aa, Homo sapiens
LHPPVPAVSR

SEQ ID NO: 116 - protein, 8 aa, Homo sapiens
AEDGLWLR

SEQ ID NO: 117 - protein, 10 aa, Homo sapiens
LHPPIPTFAR

SEQ ID NO: 118 - protein, 15 aa, Homo sapiens
VLTQLVATYPQGFVR

SEQ ID NO: 119 - protein, 14 aa, Homo sapiens
TLDFIDVLLLSEDK

SEQ ID NO: 120 - protein, 11 aa, Homo sapiens
VVLALTLLHFR

SEQ ID NO: 121 - protein, 7 aa, Homo sapiens
FNQENIK

SEQ ID NO: 122 - protein, 14 aa, Homo sapiens
TLTQLVTTYPQGFK

SEQ ID NO: 123 - protein, 7 aa, Homo sapiens
FDPENSK

SEQ ID NO: 124 - protein, 8 aa, Homo sapiens
ALSDEDIR

SEQ ID NO: 125 - protein, 11 aa, Homo sapiens
LVHDFTDAVIR

SEQ ID NO: 126 - protein, 8 aa, Homo sapiens
SYITIFNK

SEQ ID NO: 127 - protein, 10 aa, Homo sapiens
LHPPAPFISR

SEQ ID NO: 128 - protein, 9 aa, Homo sapiens
QQGAEAWLK

SEQ ID NO: 129 - protein, 8 aa, Homo sapiens
TENGLWLK

SEQ ID NO: 130 - protein, 9 aa, Homo sapiens
LLHLLGLEK

SEQ ID NO: 131 - protein, 9 aa, Homo sapiens
VVVALTLLR

SEQ ID NO: 132 - protein, 13 aa, Homo sapiens
SPLAYVPFSAGPR

SEQ ID NO: 133 - protein, 11 aa, Homo sapiens
LHHYLDFIYYR

SEQ ID NO: 134 - protein, 10 aa, Homo sapiens
QYPPVTLVSR

SEQ ID NO: 135 - protein, 12 aa, Homo sapiens
TLDFIDVLLLAR

SEQ ID NO: 136 - protein, 9 aa, Homo sapiens
FDPDNPQQR

SEQ ID NO: 137 - protein, 13 aa, Homo sapiens
HPYAYVPFSAGPR

SEQ ID NO: 138 - protein, 9 aa, Homo sapiens
FFPENAQGR

SEQ ID NO: 139 - protein, 7 aa, Homo sapiens
LSHEDIR

SEQ ID NO: 140 - protein, 10 aa, Homo sapiens
LFPSVPLFAR

SEQ ID NO: 141 - protein, 13 aa, Homo sapiens
ILHTFTNSVIAER

SEQ ID NO: 142 - protein, 13 aa, Homo sapiens
GTEAVIIPYALHR

SEQ ID NO: 143 - protein, 12 aa, Homo sapiens
YFPNPEEFQPER

SEQ ID NO: 144 - protein, 14 aa, Homo sapiens
NIGAQSNDDSEYVR

SEQ ID NO: 145 - protein, 9 aa, Homo sapiens
GLAALDGPK

SEQ ID NO: 146 - protein, 9 aa, Homo sapiens
FSQENSDQR

SEQ ID NO: 147 - protein, 10 aa, Homo sapiens
LIPAVPSISR

SEQ ID NO: 148 - protein, 13 aa, Homo sapiens
HPYAYLPFSAGSR

SEQ ID NO: 149 - protein, 7 aa, Homo sapiens
LEEIIEK

SEQ ID NO: 150 - protein, 8 aa, Homo sapiens
FSPPLLGK

SEQ ID NO: 151 - protein, 7 aa, Homo sapiens
AIFELSK

SEQ ID NO: 152 - protein, 7 aa, Homo sapiens
LSPQGYR

SEQ ID NO: 153 - protein, 13 aa, Homo sapiens
LYSLLYHSDIIFK

SEQ ID NO: 154 - protein, 11 aa, Homo sapiens
VTIALILLHFR

SEQ ID NO: 155 - protein, 8 aa, Homo sapiens
ILESWVGR

SEQ ID NO: 156 - protein, 12 aa, Homo sapiens
LLDKPITFPDGR

SEQ ID NO: 157 - protein, 7 aa, Homo sapiens
EFEVYHK

SEQ ID NO: 158 - protein, 10 aa, Homo sapiens
WEEHIAQNSR

SEQ ID NO: 159 - protein, 10 aa, Homo sapiens
FSSQGQIFSK

SEQ ID NO: 160 - protein, 9 aa, Homo sapiens
GLVTLDGSK

SEQ ID NO: 161 - protein, 12 aa, Homo sapiens
DFSEADLQAEVK

SEQ ID NO: 162 - protein, 11 aa, Homo sapiens
FNQELHQFTEK

SEQ ID NO: 163 - protein, 13 aa, Homo sapiens
LAPDHSRPPQPVR

SEQ ID NO: 164 - protein, 9 aa, Homo sapiens
VAVALTLLR

SEQ ID NO: 165 - protein, 14 aa, Homo sapiens
IHPYAFIPFSAGLR

SEQ ID NO: 166 - protein, 8 aa, Homo sapiens
LFAIHEIK

SEQ ID NO: 167 - protein, 13 aa, Homo sapiens
YVHFITNPLSYHK

SEQ ID NO: 168 - protein, 7 aa, Homo sapiens
YLDENGK

SEQ ID NO: 169 - protein, 15 aa, Homo sapiens
AGLGILPPLNDIEFK

SEQ ID NO: 170 - protein, 11 aa, Homo sapiens
AHILNNLDNFK

SEQ ID NO: 171 - protein, 9 aa, Homo sapiens
LSSASLNIR

SEQ ID NO: 172 - protein, 9 aa, Homo sapiens
RPGEPPLIK

SEQ ID NO: 173 - protein, 13 aa, Homo sapiens
QHGDTFTVLLGGK

SEQ ID NO: 174 - protein, 15 aa, Homo sapiens
LLFGIQYPDSDVLFR

SEQ ID NO: 175 - protein, 16 aa, Homo sapiens
YITFILDPFQYQLVIK

SEQ ID NO: 176 - protein, 9 aa, Homo sapiens
AFSISQLQK

SEQ ID NO: 177 - protein, 9 aa, Homo sapiens
LSSYSTTIR

SEQ ID NO: 178 - protein, 9 aa, Homo sapiens
QVFEPQLLK

SEQ ID NO: 179 - protein, 8 aa, Homo sapiens
FFALSEVK

SEQ ID NO: 180 - protein, 7 aa, Homo sapiens
AAPTLLR

SEQ ID NO: 181 - protein, 7 aa, Homo sapiens
EWLEVGR

SEQ ID NO: 182 - protein, 11 aa, Homo sapiens
EEATQVLGEAR

SEQ ID NO: 183 - protein, 9 aa, Homo sapiens
LVHEDYTLK

SEQ ID NO: 184 - protein, 9 aa, Homo sapiens
FVYSLLWPR

SEQ ID NO: 185 - protein, 9 aa, Homo sapiens
RPWEPPLDK

SEQ ID NO: 186 - protein, 11 aa, Homo sapiens
LQQVDSLHPHR

SEQ ID NO: 187 - protein, 9 aa, Homo sapiens
NPNVQQALR

SEQ ID NO: 188 - protein, 13 aa, Homo sapiens
HLQVETLTQEDIK

SEQ ID NO: 189 - protein, 7 aa, Homo sapiens
AQALGTR

SEQ ID NO: 190 - protein, 13 aa, Homo sapiens
LNPEVLSPNAVQR

SEQ ID NO: 191 - protein, 10 aa, Homo sapiens
NPALFPRPER

SEQ ID NO: 192 - protein, 9 aa, Homo sapiens
AELSLEAIK

SEQ ID NO: 193 - protein, 9 aa, Homo sapiens
LNPDVLSPK

SEQ ID NO: 194 - protein, 7 aa, Homo sapiens
YNLGGPR

SEQ ID NO: 195 - protein, 10 aa, Homo sapiens
NAALFPRPER

SEQ ID NO: 196 - protein, 16 aa, Homo sapiens
GTEVIINLWALHHNEK

SEQ ID NO: 197 - protein, 12 aa, Homo sapiens
TTVIVGHHQLAK

SEQ ID NO: 198 - protein, 8 aa, Homo sapiens
EAQAEGST

SEQ ID NO: 199 - protein, 14 aa, Homo sapiens
ANVDSSIGEFAVDK

SEQ ID NO: 200 - protein, 11 aa, Homo sapiens
DSLVDLVPWLK

SEQ ID NO: 201 - protein, 11 aa, Homo sapiens
IPLDESAIVVK

SEQ ID NO: 202 - protein, 9 aa, Homo sapiens
EIQTVIGER

SEQ ID NO: 203 - protein, 13 aa, Homo sapiens
VWISGEETLIISK

SEQ ID NO: 204 - protein, 9 aa, Homo sapiens
ALSGPGLVR

SEQ ID NO: 205 - protein, 12 aa, Homo sapiens
IHDLSLHPDETK

SEQ ID NO: 206 - protein, 10 aa, Homo sapiens
HPNVEEAIIK

SEQ ID NO: 207 - protein, 12 aa, Homo sapiens
GIIFNNNPELWK

SEQ ID NO: 208 - protein, 7 aa, Homo sapiens
LLWNWWK

SEQ ID NO: 209 - protein, 8 aa, Homo sapiens
SALLLGIR

SEQ ID NO: 210 - protein, 9 aa, Homo sapiens
ESLVAGQWR

SEQ ID NO: 211 - protein, 11 aa, Homo sapiens
ATVLGEYALPK

SEQ ID NO: 212 - protein, 7 aa, Homo sapiens
LEIKPWK

SEQ ID NO: 213 - protein, 7 aa, Homo sapiens
NPQVQQK

SEQ ID NO: 214 - protein, 10 aa, Homo sapiens
SLAAFLQQLR

SEQ ID NO: 215 - protein, 12 aa, Homo sapiens
GHVEDLYSELNK

SEQ ID NO: 216 - protein, 13 aa, Homo sapiens
LVTSTAYTSPQPR

SEQ ID NO: 217 - protein, 16 aa, Homo sapiens
NLFSLPIDVPFSGLYR

SEQ ID NO: 218 - protein, 11 aa, Homo sapiens
LVSVHWPASVR

SEQ ID NO: 219 - protein, 8 aa, Homo sapiens
FTHFHGEI

SEQ ID NO: 220 - protein, 8 aa, Homo sapiens
IFTVELAR

SEQ ID NO: 221 - protein, 11 aa, Homo sapiens
FSFIPFGGGLR

SEQ ID NO: 222 - protein, 10 aa, Homo sapiens
LNPPVPGGFR

SEQ ID NO: 223 - protein, 10 aa, Homo sapiens
THLFGRPTVR

SEQ ID NO: 224 - protein, 11 aa, Homo sapiens
TFELNGYQIPK

SEQ ID NO: 225 - protein, 13 aa, Homo sapiens
DALQLLIEHSWER

SEQ ID NO: 226 - protein, 14 aa, Homo sapiens
TSPTVYPVDNLPAR

SEQ ID NO: 227 - protein, 16 aa, Homo sapiens
ITLVPVLHPVDGLSVK

SEQ ID NO: 228 - protein, 13 aa, Homo sapiens
IFSHEALESYLPK

SEQ ID NO: 229 - protein, 10 aa, Homo sapiens
DTHDTAPVFK

SEQ ID NO: 230 - protein, 9 aa, Homo sapiens
DVNVFDPDR

SEQ ID NO: 231 - protein, 10 aa, Homo sapiens
THLLGRPLIR

SEQ ID NO: 232 - protein, 8 aa, Homo sapiens
LDTLSGLR

SEQ ID NO: 233 - protein, 15 aa, Homo sapiens
TVLQTFELDGFQIPK

SEQ ID NO: 234 - protein, 11 aa, Homo sapiens
FHYLPFGGGVR

SEQ ID NO: 235 - protein, 10 aa, Homo sapiens
IQLVIQDTLR

SEQ ID NO: 236 - protein, 11 aa, Homo sapiens
VLAVELASTSR

SEQ ID NO: 237 - protein, 7 aa, Homo sapiens
HPTVLEK

SEQ ID NO: 238 - protein, 10 aa, Homo sapiens
LFTPISGGYR

SEQ ID NO: 239 - protein, 10 aa, Homo sapiens
EELVAQGLGR

SEQ ID NO: 240 - protein, 16 aa, Homo sapiens
AAEPGDALDLIIHSAR

SEQ ID NO: 241 - protein, 11 aa, Homo sapiens
TFELDGYQIPK

SEQ ID NO: 242 - protein, 8 aa, Homo sapiens
TILLGEHR

SEQ ID NO: 243 - protein, 10 aa, Homo sapiens
SPPEGFDPER

SEQ ID NO: 244 - protein, 10 aa, Homo sapiens
THLLGRPVIR

SEQ ID NO: 245 - protein, 10 aa, Homo sapiens
DTHETAAVYR

SEQ ID NO: 246 - protein, 8 aa, Homo sapiens
ASTLPLPK

SEQ ID NO: 247 - protein, 10 aa, Homo sapiens
LLPPVSGGYR

SEQ ID NO: 248 - protein, 9 aa, Homo sapiens
HLEGAISEK

SEQ ID NO: 249 - protein, 8 aa, Homo sapiens
VSGAENVR

SEQ ID NO: 250 - protein, 11 aa, Homo sapiens
AAIPAALPSDK

SEQ ID NO: 251 - protein, 7 aa, Homo sapiens
SLEEIPR

SEQ ID NO: 252 - protein, 8 aa, Homo sapiens
NSQPATPR

SEQ ID NO: 253 - protein, 11 aa, Homo sapiens
EIEVDGFLFPK

SEQ ID NO: 254 - protein, 15 aa, Homo sapiens
DPTAFSEPESFQPHR

SEQ ID NO: 255 - protein, 11 aa, Homo sapiens
VVLAPETGELK

SEQ ID NO: 256 - protein, 11 aa, Homo sapiens
SIPEDTVTFVR

SEQ ID NO: 257 - protein, 11 aa, Homo sapiens
DIHVGDYIIPK

SEQ ID NO: 258 - protein, 15 aa, Homo sapiens
TVYVAAPALVEELLR

SEQ ID NO: 259 - protein, 9 aa, Homo sapiens
GTGPPALVR

SEQ ID NO: 260 - protein, 9 aa, Homo sapiens
HLVPGPWGR

SEQ ID NO: 261 - protein, 15 aa, Homo sapiens
SLLAPLLLRPQAAAR

SEQ ID NO: 262 - protein, 14 aa, Homo sapiens
DLESGAHLTHFLFR

SEQ ID NO: 263 - protein, 12 aa, Homo sapiens
WAPELGASLGYR

SEQ ID NO: 264 - protein, 9 aa, Homo sapiens
SINLQFLDR

SEQ ID NO: 265 - protein, 14 aa, Homo sapiens
FGLEGIAAVLLGSR

SEQ ID NO: 266 - protein, 10 aa, Homo sapiens
HPEVQQTVYR

SEQ ID NO: 267 - protein, 14 aa, Homo sapiens
VDNFGSIPFGHGVR

SEQ ID NO: 268 - protein, 9 aa, Homo sapiens
FSQIHVDNK

SEQ ID NO: 269 - protein, 11 aa, Homo sapiens
TSSQTNAVHAK

SEQ ID NO: 270 - protein, 10 aa, Homo sapiens
LFPVLPGNGR

SEQ ID NO: 271 - protein, 14 aa, Homo sapiens
THGLLTPGGPIHVR

SEQ ID NO: 272 - protein, 7 aa, Homo sapiens
SWDGLFK

SEQ ID NO: 273 - protein, 15 aa, Homo sapiens
VTQEDLVIGGYLIPK

SEQ ID NO: 274 - protein, 11 aa, Homo sapiens
WLRPFIPKPWR

SEQ ID NO: 275 - protein, 10 aa, Homo sapiens
HVPTAADVPK

SEQ ID NO: 276 - protein, 13 aa, Homo sapiens
VSEDDLENLLLIK

SEQ ID NO: 277 - protein, 9 aa, Homo sapiens
FGPGAPKPR

SEQ ID NO: 278 - protein, 11 aa, Homo sapiens
FTYFPFSLGHR

SEQ ID NO: 279 - protein, 11 aa, Homo sapiens
AEQLVEILEAK

SEQ ID NO: 280 - protein, 9 aa, Homo sapiens
ALQTVFGER

SEQ ID NO: 281 - protein, 10 aa, Homo sapiens
LYPPAWGTFR

SEQ ID NO: 282 - protein, 7 aa, Homo sapiens
QPEIVAR

SEQ ID NO: 283 - protein, 12 aa, Homo sapiens
LLEEETLIDGVR

SEQ ID NO: 284 - protein, 9 aa, Homo sapiens
LQYLSQVLK

SEQ ID NO: 285 - protein, 7 aa, Homo sapiens
DLNLLDR

SEQ ID NO: 286 - protein, 7 aa, Homo sapiens
LDFNPDR

SEQ ID NO: 287 - protein, 8 aa, Homo sapiens
LTTPVFGK

SEQ ID NO: 288 - protein, 8 aa, Homo sapiens
QHVSIIEK

SEQ ID NO: 289 - protein, 9 aa, Homo sapiens
SGLNIAHFK

SEQ ID NO: 290 - protein, 10 aa, Homo sapiens
FAYVPFGAGR

SEQ ID NO: 291 - protein, 11 aa, Homo sapiens
YLQDNPASGEK

SEQ ID NO: 292 - protein, 12 aa, Homo sapiens
NEDLNAEDVYSR

SEQ ID NO: 293 - protein, 12 aa, Homo sapiens
SPIEFLENAYEK

SEQ ID NO: 294 - protein, 12 aa, Homo sapiens
EYFESWGESGEK

SEQ ID NO: 295 - protein, 14 aa, Homo sapiens
LAAGHLVQLPAGVK

SEQ ID NO: 296 - protein, 14 aa, Homo sapiens
IDDILQTLLDATYK

SEQ ID NO: 297 - protein, 16 aa, Homo sapiens
GHEIVVLAPDASLYIR

SEQ ID NO: 298 - protein, 11 aa, Homo sapiens
YLSIPTVFFLR

SEQ ID NO: 299 - protein, 17 aa, Homo sapiens
EVSVVDILSHASVWLFR

SEQ ID NO: 300 - protein, 11 aa, Homo sapiens
YLSIPAVFFWR

SEQ ID NO: 301 - protein, 14 aa, Homo sapiens
FFTLTAYAVPWTQK

SEQ ID NO: 302 - protein, 11 aa, Homo sapiens
YLSIPAVFFLR

SEQ ID NO: 303 - protein, 9 aa, Homo sapiens
DIVEVLSDR

SEQ ID NO: 304 - protein, 10 aa, Homo sapiens
SPDPVSYIPR

SEQ ID NO: 305 - protein, 11 aa, Homo sapiens
SFLTAPQTEYR

SEQ ID NO: 306 - protein, 6 aa, Homo sapiens
WTAPLR

SEQ ID NO: 307 - protein, 13 aa, Homo sapiens
TYSTSYTLEDQDR

SEQ ID NO: 308 - protein, 11 aa, Homo sapiens
YFSLPSVVFTR

SEQ ID NO: 309 - protein, 7 aa, Homo sapiens
AEIWLIR

SEQ ID NO: 310 - protein, 14 aa, Homo sapiens
ANLIASALAQIPQK

SEQ ID NO: 311 - protein, 10 aa, Homo sapiens
ANVIASALAK

SEQ ID NO: 312 - protein, 7 aa, Homo sapiens
ADVWLIR

SEQ ID NO: 313 - protein, 9 aa, Homo sapiens
TILDELIQR

SEQ ID NO: 314 - protein, 6 aa, Homo sapiens
NSWNFK

SEQ ID NO: 315 - protein, 10 aa, Homo sapiens
FEVYPTSLTK

SEQ ID NO: 316 - protein, 9 aa, Homo sapiens
SVINDPIYK

SEQ ID NO: 317 - protein, 12 aa, Homo sapiens
WIPQNDLLGLPK

SEQ ID NO: 318 - protein, 7 aa, Homo sapiens
LHYFNAR

SEQ ID NO: 319 - protein, 7 aa, Homo sapiens
LALIQEK

SEQ ID NO: 320 - protein, 7 aa, Homo sapiens
LHYFNGR

SEQ ID NO: 321 - protein, 7 aa, Homo sapiens
LHYPNGR

SEQ ID NO: 322 - protein, 7 aa, Homo sapiens
YFPVFEK

SEQ ID NO: 323 - protein, 8 aa, Homo sapiens
TVYNIFRP

SEQ ID NO: 324 - protein, 7 aa, Homo sapiens
LHYSNAR

SEQ ID NO: 325 - protein, 11 aa, Homo sapiens
HHLQIPIHFPK

SEQ ID NO: 326 - protein, 7 aa, Homo sapiens
GLAHAIR

SEQ ID NO: 327 - protein, 8 aa, Homo sapiens
QPWFLGDK

SEQ ID NO: 328 - protein, 9 aa, Homo sapiens
FLPRPVFTK

SEQ ID NO: 329 - protein, 7 aa, Homo sapiens
SQWLDVK

SEQ ID NO: 330 - protein, 9 aa, Homo sapiens
FLPKPLYTR

SEQ ID NO: 331 - protein, 6 aa, Homo sapiens
SSQFLR

SEQ ID NO: 332 - protein, 7 aa, Homo sapiens
TLGLYGK

SEQ ID NO: 333 - protein, 8 aa, Homo sapiens
NDIPFELR

SEQ ID NO: 334 - protein, 10 aa, Homo sapiens
DFPPADPTIK

SEQ ID NO: 335 - protein, 8 aa, Homo sapiens
NGIPLELR

SEQ ID NO: 336 - protein, 6 aa, Homo sapiens
LFVGAR

SEQ ID NO: 337 - protein, 14 aa, Homo sapiens
VTPPAVTGSPEFER

SEQ ID NO: 338 - protein, 8 aa, Homo sapiens
SGLGSGPK

SEQ ID NO: 339 - protein, 6 aa, Homo sapiens
NTPAPR

SEQ ID NO: 340 - protein, 6 aa, Homo sapiens
APIYVR

SEQ ID NO: 341 - protein, 9 aa, Homo sapiens
NLNDEILDR

SEQ ID NO: 342 - protein, 7 aa, Homo sapiens
GTVGDWK

SEQ ID NO: 343 - protein, 7 aa, Homo sapiens
HAFLELK

SEQ ID NO: 344 - protein, 7 aa, Homo sapiens
LTFHFQF

SEQ ID NO: 345 - protein, 7 aa, Homo sapiens
EDVIFNR

SEQ ID NO: 346 - protein, 7 aa, Homo sapiens
GVSGDWK

SEQ ID NO: 347 - protein, 10 aa, Homo sapiens
DPGTPDQFLR

SEQ ID NO: 348 - protein, 8 aa, Homo sapiens
YFEFHGVR

SEQ ID NO: 349 - protein, 7 aa, Homo sapiens
QQFLILR

SEQ ID NO: 350 - protein, 7 aa, Homo sapiens
YSLDVWS

SEQ ID NO: 351 - protein, 7 aa, Homo sapiens
ESSFVEK

SEQ ID NO: 352 - protein, 8 aa, Homo sapiens
EELAQFHR

SEQ ID NO: 353 - protein, 9 aa, Homo sapiens
IQTLTSSVR

SEQ ID NO: 354 - protein, 9 aa, Homo sapiens
YYSIASSSK

SEQ ID NO: 355 - protein, 9 aa, Homo sapiens
FAVFGLGNK

SEQ ID NO: 356 - protein, 12 aa, Homo sapiens
SYENQKPPFDAK

SEQ ID NO: 357 - protein, 13 aa, Homo sapiens
DGALTQLNVAFSR

SEQ ID NO: 358 - protein, 14 aa, Homo sapiens
TALTYYLDITNPPR

SEQ ID NO: 359 - protein, 14 aa, Homo sapiens
QYELVVHTDIDAAK

SEQ ID NO: 360 - protein, 10 aa, Homo sapiens
EWLLAHEGHR

SEQ ID NO: 361 - protein, 9 aa, Homo sapiens
TTLDSPLGK

SEQ ID NO: 362 - protein, 9 aa, Homo sapiens
AQLASLNGK

SEQ ID NO: 363 - protein, 8 aa, Homo sapiens
STFVNILK

SEQ ID NO: 364 - protein, 7 aa, Homo sapiens
AVSEGYK

SEQ ID NO: 365 - protein, 10 aa, Homo sapiens
DVVVSQSQLR

SEQ ID NO: 366 - protein, 8 aa, Homo sapiens
GFLEALGR

SEQ ID NO: 367 - protein, 9 aa, Homo sapiens
AVNPNHSLR

SEQ ID NO: 368 - protein, 8 aa, Homo sapiens
APSIDEPR

SEQ ID NO: 369 - protein, 8 aa, Homo sapiens
GFLEDLGR

SEQ ID NO: 370 - protein, 9 aa, Homo sapiens
LPSSSTSSR

SEQ ID NO: 371 - protein, 9 aa, Homo sapiens
IYEQEGLEK

SEQ ID NO: 372 - protein, 9 aa, Homo sapiens
DSQPLEYSR

SEQ ID NO: 373 - protein, 7 aa, Homo sapiens
DPIGYLK

SEQ ID NO: 374 - protein, 11 aa, Homo sapiens
GIYPASTQLDR

SEQ ID NO: 375 - protein, 10 aa, Homo sapiens
VFLATNSDYK

SEQ ID NO: 376 - protein, 10 aa, Homo sapiens
QAGVYHPNVK

SEQ ID NO: 377 - protein, 7 aa, Homo sapiens
VDELLEK

SEQ ID NO: 378 - protein, 9 aa, Homo sapiens
EGYENFFDK

SEQ ID NO: 379 - protein, 9 aa, Homo sapiens
GELIHVFNK

SEQ ID NO: 380 - protein, 9 aa, Homo sapiens
NTEYFNQLK

SEQ ID NO: 381 - protein, 12 aa, Homo sapiens
SHGLLVQQALPK

SEQ ID NO: 382 - protein, 7 aa, Homo sapiens
IGYLNDR

SEQ ID NO: 383 - protein, 12 aa, Homo sapiens
TVVLGDLLIDDK

SEQ ID NO: 384 - protein, 14 aa, Homo sapiens
YFGPDFLEQIVLTR

SEQ ID NO: 385 - protein, 12 aa, Homo sapiens
GAAGGLGLAGGR

SEQ ID NO: 386 - protein, 9 aa, Homo sapiens
FPILSANIK

SEQ ID NO: 387 - protein, 11 aa, Homo sapiens
VPVVQAYAFGK

SEQ ID NO: 388 - protein, 8 aa, Homo sapiens
VPSYDPLK

SEQ ID NO: 389 - protein, 10 aa, Homo sapiens
HPDYAILAAR

SEQ ID NO: 390 - protein, 9 aa, Homo sapiens
LNSAIIYDR

SEQ ID NO: 391 - protein, 10 aa, Homo sapiens
STLDIVLANK

SEQ ID NO: 392 - protein, 10 aa, Homo sapiens
SNQQNLGTIK

SEQ ID NO: 393 - protein, 11 aa, Homo sapiens
ENTPPALSGTR

SEQ ID NO: 394 - protein, 9 aa, Homo sapiens
IFQEPTEPK

SEQ ID NO: 395 - protein, 8 aa, Homo sapiens
FLIDGFPR

SEQ ID NO: 396 - protein, 8 aa, Homo sapiens
LIIQAGIK

SEQ ID NO: 397 - protein, 7 aa, Homo sapiens
FPSINHD

SEQ ID NO: 398 - protein, 12 aa, Homo sapiens
GIIASSVGTILK

SEQ ID NO: 399 - protein, 8 aa, Homo sapiens
IQAIAWAR

SEQ ID NO: 400 - protein, 8 aa, Homo sapiens
IYQYVINK

SEQ ID NO: 401 - protein, 10 aa, Homo sapiens
FSDSYASVIK

SEQ ID NO: 402 - protein, 10 aa, Homo sapiens
FVGQDVEGER

SEQ ID NO: 403 - protein, 7 aa, Homo sapiens
LSHYLQK

SEQ ID NO: 404 - protein, 8 aa, Homo sapiens
DNNLTTGK

SEQ ID NO: 405 - protein, 7 aa, Homo sapiens
LDLPIER

SEQ ID NO: 406 - protein, 9 aa, Homo sapiens
ITETEDPVK

SEQ ID NO: 407 - protein, 7 aa, Homo sapiens
VPVDGNK

SEQ ID NO: 408 - protein, 8 aa, Homo sapiens
DNNITTGK

SEQ ID NO: 409 - protein, 13 aa, Homo sapiens
ADGILVPGGFGIR

SEQ ID NO: 410 - protein, 9 aa, Homo sapiens
FEVNPNLIK

SEQ ID NO: 411 - protein, 8 aa, Homo sapiens
FLDINLYK

SEQ ID NO: 412 - protein, 12 aa, Homo sapiens
GIIASSIGTILK

SEQ ID NO: 413 - protein, 9 aa, Mus musculus
YTVNDLVLR

SEQ ID NO: 414 - protein, 10 aa, Mus musculus
DPGFFPNPNK

SEQ ID NO: 415 - protein, 10 aa, Mus musculus
GAGTSTSSPR

SEQ ID NO: 416 - protein, 10 aa, Mus musculus
LHPISVTLQR

SEQ ID NO: 417 - protein, 9 aa, Mus musculus
YLGFGWGVR

SEQ ID NO: 418 - protein, 13 aa, Mus musculus
ADEYTQNFYWDLR

SEQ ID NO: 419 - protein, 12 aa, Mus musculus
DHAAAWDVIFNK

SEQ ID NO: 420 - protein, 14 aa, Mus musculus
FSFESISSVIFGER

SEQ ID NO: 421 - protein, 13 aa, Mus musculus
LGTLESVYIVDPK

SEQ ID NO: 422 - protein, 7 aa, Mus musculus
IEVQNLR

SEQ ID NO: 423 - protein, 9 aa, Mus musculus
VVFLIDPFK

SEQ ID NO: 424 - protein, 9 aa, Mus musculus
FDFDVSDDK

SEQ ID NO: 425 - protein, 10 aa, Mus musculus
EIDQYVGFSR

SEQ ID NO: 426 - protein, 8 aa, Mus musculus
DLSTLIFK

SEQ ID NO: 427 - protein, 7 aa, Mus musculus
TPSFNDR

SEQ ID NO: 428 - protein, 8 aa, Mus musculus
YGPIYSLR

SEQ ID NO: 429 - protein, 14 aa, Mus musculus
ANIDSSIGEFAIPK

SEQ ID NO: 430 - protein, 9 aa, Mus musculus
ALTGPGLVR

SEQ ID NO: 431 - protein, 10 aa, Mus musculus
YFQPFGFGPR

SEQ ID NO: 432 - protein, 9 aa, Mus musculus
EIHTVVGDR

SEQ ID NO: 433 - protein, 13 aa, Mus musculus
VWISGEETLIISK

SEQ ID NO: 434 - protein, 10 aa, Mus musculus
HLVEIIFSPR

SEQ ID NO: 435 - protein, 8 aa, Mus musculus
IEDIQNLK

SEQ ID NO: 436 - protein, 7 aa, Mus musculus
HSHYISR

SEQ ID NO: 437 - protein, 11 aa, Mus musculus
DTSLNGFYIPK

SEQ ID NO: 438 - protein, 8 aa, Mus musculus
EANYLVSK

SEQ ID NO: 439 - protein, 12 aa, Mus musculus
TFNDNFVLFLQK

SEQ ID NO: 440 - protein, 10 aa, Mus musculus
DNGGLIPEEK

SEQ ID NO: 441 - protein, 11 aa, Mus musculus
TVQEHYQDFNK

SEQ ID NO: 442 - protein, 11 aa, Mus musculus
DTSLNGFHIPK

SEQ ID NO: 443 - protein, 8 aa, Mus musculus
EANHLVSK

SEQ ID NO: 444 - protein, 9 aa, Mus musculus
LELFVVLAR

SEQ ID NO: 445 - protein, 9 aa, Mus musculus
TIEEALIQK

SEQ ID NO: 446 - protein, 10 aa, Mus musculus
LGPGSQLLYR

SEQ ID NO: 447 - protein, 11 aa, Mus musculus
ASSISGYDIPK

SEQ ID NO: 448 - protein, 11 aa, Mus musculus
AQAGTPVAIHK

SEQ ID NO: 449 - protein, 10 aa, Mus musculus
LTPSVPFTTR

SEQ ID NO: 450 - protein, 7 aa, Mus musculus
NPQVQQR

SEQ ID NO: 451 - protein, 10 aa, Mus musculus
IQDLYSELNK

SEQ ID NO: 452 - protein, 14 aa, Mus musculus
DTEEEALTFIAAIK

SEQ ID NO: 453 - protein, 8 aa, Mus musculus
TESAHPQR

SEQ ID NO: 454 - protein, 7 aa, Mus musculus
DLGQPPR

SEQ ID NO: 455 - protein, 14 aa, Mus musculus
VWQAHTLAWDTIFK

SEQ ID NO: 456 - protein, 10 aa, Mus musculus
LNPPVPGGFR

SEQ ID NO: 457 - protein, 11 aa, Mus musculus
FSFIPFGGGLR

SEQ ID NO: 458 - protein, 11 aa, Mus musculus
TFELNGYQIPK

SEQ ID NO: 459 - protein, 11 aa, Mus musculus
LVSVHWPASVR

SEQ ID NO: 460 - protein, 8 aa, Mus musculus
IFTVELAR

SEQ ID NO: 461 - protein, 14 aa, Mus musculus
TSPTVYPVDNLPAR

SEQ ID NO: 462 - protein, 7 aa, Mus musculus
EEFNPDR

SEQ ID NO: 463 - protein, 11 aa, Mus musculus
FIVPHPEDASR

SEQ ID NO: 464 - protein, 13 aa, Mus musculus
DALQLLIEHSWER

SEQ ID NO: 465 - protein, 12 aa, Mus musculus
AQLQETGPDGVR

SEQ ID NO: 466 - protein, 10 aa, Mus musculus
LYPVVPTNSR

SEQ ID NO: 467 - protein, 10 aa, Mus musculus
EVTGVVPFGK

SEQ ID NO: 468 - protein, 11 aa, Mus musculus
ETEINGFLFPK

SEQ ID NO: 469 - protein, 10 aa, Mus musculus
SPEIQEALHK

SEQ ID NO: 470 - protein, 7 aa, Mus musculus
WTLLDPR

SEQ ID NO: 471 - protein, 9 aa, Mus musculus
SINLQFVDR

SEQ ID NO: 472 - protein, 10 aa, Mus musculus
LYPVVPGNSR

SEQ ID NO: 473 - protein, 13 aa, Mus musculus
YGPIWSGSFGTLR

SEQ ID NO: 474 - protein, 7 aa, Mus musculus
TVLVPER

SEQ ID NO: 475 - protein, 7 aa, Mus musculus
GSESVLR

SEQ ID NO: 476 - protein, 14 aa, Mus musculus
FGLESIGAVLLGSR

SEQ ID NO: 477 - protein, 14 aa, Mus musculus
IHLPLQLDASLGSR

SEQ ID NO: 478 - protein, 11 aa, Mus musculus
LHELQVHGAAR

SEQ ID NO: 479 - protein, 8 aa, Mus musculus
SFSIATLR

SEQ ID NO: 480 - protein, 11 aa, Mus musculus
GYGVVFSSGER

SEQ ID NO: 481 - protein, 13 aa, Mus musculus
EALVNQAEAFSGR

SEQ ID NO: 482 - protein, 12 aa, Mus musculus
FTDLAPIGLPHK

SEQ ID NO: 483 - protein, 12 aa, Mus musculus
TPPQYQIHFLSR

SEQ ID NO: 484 - protein, 11 aa, Mus musculus
GFGVVFSNGNR

SEQ ID NO: 485 - protein, 11 aa, Mus musculus
FINLVPNNIPR

SEQ ID NO: 486 - protein, 11 aa, Mus musculus
VQEEIDHVIGR

SEQ ID NO: 487 - protein, 13 aa, Mus musculus
EALIDHGEEFSDR

SEQ ID NO: 488 - protein, 14 aa, Mus musculus
INNGLGIVFSNGNR

SEQ ID NO: 489 - protein, 13 aa, Mus musculus
EALIDQGDEFSDK

SEQ ID NO: 490 - protein, 11 aa, Mus musculus
IQEEIAHVIGR

SEQ ID NO: 491 - protein, 8 aa, Mus musculus
TDSSLLSR

SEQ ID NO: 492 - protein, 10 aa, Mus musculus
FDPGHFLDEK

SEQ ID NO: 493 - protein, 11 aa, Mus musculus
FGDIAPLNLPR

SEQ ID NO: 494 - protein, 8 aa, Mus musculus
FQDFFIPK

SEQ ID NO: 495 - protein, 10 aa, Mus musculus
LNSFIALVNK

SEQ ID NO: 496 - protein, 10 aa, Mus musculus
GNPESSFNDK

SEQ ID NO: 497 - protein, 12 aa, Mus musculus
GVILAPYGPEWR

SEQ ID NO: 498 - protein, 10 aa, Mus musculus
TTWDPVQAPR

SEQ ID NO: 499 - protein, 14 aa, Mus musculus
SFIAILDNLLTENR

SEQ ID NO: 500 - protein, 7 aa, Mus musculus
YPEIEEK

SEQ ID NO: 501 - protein, 10 aa, Mus musculus
SQDLLTSLTK

SEQ ID NO: 502 - protein, 13 aa, Mus musculus
LLTIIHFINDNFK

SEQ ID NO: 503 - protein, 11 aa, Mus musculus
GNGIAFSDGER

SEQ ID NO: 504 - protein, 12 aa, Mus musculus
EHQDSLDPNSPR

SEQ ID NO: 505 - protein, 7 aa, Mus musculus
FDYDDER

SEQ ID NO: 506 - protein, 13 aa, Mus musculus
LPFVGNFFQIDTK

SEQ ID NO: 507 - protein, 12 aa, Mus musculus
NGLVVSNGQTWK

SEQ ID NO: 508 - protein, 12 aa, Mus musculus
EEGGQPFNPHLK

SEQ ID NO: 509 - protein, 8 aa, Mus musculus
FNGFHLPK

SEQ ID NO: 510 - protein, 7 aa, Mus musculus
EVTVDTK

SEQ ID NO: 511 - protein, 13 aa, Mus musculus
IQEETHHLVEAIR

SEQ ID NO: 512 - protein, 11 aa, Mus musculus
NADVVYDFLSR

SEQ ID NO: 513 - protein, 7 aa, Mus musculus
GGPFDLK

SEQ ID NO: 514 - protein, 9 aa, Mus musculus
DVVDAFLLK

SEQ ID NO: 515 - protein, 8 aa, Mus musculus
GTLATLDK

SEQ ID NO: 516 - protein, 13 aa, Mus musculus
LPYTDAVLHEAQR

SEQ ID NO: 517 - protein, 8 aa, Mus musculus
YALLLLLR

SEQ ID NO: 518 - protein, 7 aa, Mus musculus
YPQVQQR

SEQ ID NO: 519 - protein, 10 aa, Mus musculus
QDPGTEFTEK

SEQ ID NO: 520 - protein, 11 aa, Mus musculus
EELIQELGPGR

SEQ ID NO: 521 - protein, 13 aa, Mus musculus
EALGGQAEEFSGR

SEQ ID NO: 522 - protein, 9 aa, Mus musculus
VWPTGDQSR

SEQ ID NO: 523 - protein, 8 aa, Mus musculus
FQTSGPAR

SEQ ID NO: 524 - protein, 7 aa, Mus musculus
APSLSDR

SEQ ID NO: 525 - protein, 7 aa, Mus musculus
FLDEDGR

SEQ ID NO: 526 - protein, 14 aa, Mus musculus
TFDGHGVFFANGER

SEQ ID NO: 527 - protein, 7 aa, Mus musculus
EFYQQFK

SEQ ID NO: 528 - protein, 7 aa, Mus musculus
LYPIANR

SEQ ID NO: 529 - protein, 13 aa, Mus musculus
QPVLAITDPDIIK

SEQ ID NO: 530 - protein, 13 aa, Mus musculus
QGLLQPENPLLLK

SEQ ID NO: 531 - protein, 12 aa, Mus musculus
DVISFFTTSVER

SEQ ID NO: 532 - protein, 12 aa, Mus musculus
TDVEINGLFIPK

SEQ ID NO: 533 - protein, 7 aa, Mus musculus
ETEIPLK

SEQ ID NO: 534 - protein, 7 aa, Mus musculus
LYPIAGR

SEQ ID NO: 535 - protein, 13 aa, Mus musculus
ALLSPTFTSGNLK

SEQ ID NO: 536 - protein, 7 aa, Mus musculus
LYPITNR

SEQ ID NO: 537 - protein, 7 aa, Mus musculus
LYPIAIR

SEQ ID NO: 538 - protein, 7 aa, Mus musculus
FALISIK

SEQ ID NO: 539 - protein, 9 aa, Mus musculus
ALQTVFGER

SEQ ID NO: 540 - protein, 9 aa, Mus musculus
LQYLSQVLK

SEQ ID NO: 541 - protein, 12 aa, Mus musculus
LLEEETLIDGVR

SEQ ID NO: 542 - protein, 11 aa, Mus musculus
AEQLVEILEAK

SEQ ID NO: 543 - protein, 10 aa, Mus musculus
LYPPAWGTFR

SEQ ID NO: 544 - protein, 7 aa, Mus musculus
QPEIVAR

SEQ ID NO: 545 - protein, 11 aa, Mus musculus
FTYFPFSLGHR

SEQ ID NO: 546 - protein, 10 aa, Mus musculus
VLLYDPDYVK

SEQ ID NO: 547 - protein, 10 aa, Mus musculus
LYPPVISVSR

SEQ ID NO: 548 - protein, 9 aa, Mus musculus
NSLSDEDLR

SEQ ID NO: 549 - protein, 11 aa, Mus musculus
ELQQILIWVEK

SEQ ID NO: 550 - protein, 7 aa, Mus musculus
IPVPIAR

SEQ ID NO: 551 - protein, 10 aa, Mus musculus
ALDSFPGPPK

SEQ ID NO: 552 - protein, 7 aa, Mus musculus
VPQLILR

SEQ ID NO: 553 - protein, 9 aa, Mus musculus
FEFSPDPSK

SEQ ID NO: 554 - protein, 12 aa, Mus musculus
HWLFGHALEIQK

SEQ ID NO: 555 - protein, 10 aa, Mus musculus
ILIQIYAAYR

SEQ ID NO: 556 - protein, 12 aa, Mus musculus
TLPDQGLDDFLK

SEQ ID NO: 557 - protein, 13 aa, Mus musculus
TLLLLGASWILAR

SEQ ID NO: 558 - protein, 8 aa, Mus musculus
LISDGSAR

SEQ ID NO: 559 - protein, 10 aa, Mus musculus
VFPSVPLFAR

SEQ ID NO: 560 - protein, 12 aa, Mus musculus
AENVEVILTSSK

SEQ ID NO: 561 - protein, 13 aa, Mus musculus
LWIGPVPLVALYK

SEQ ID NO: 562 - protein, 9 aa, Mus musculus
FFPENSQGR

SEQ ID NO: 563 - protein, 13 aa, Mus musculus
HPYAYVPFSAGPR

SEQ ID NO: 564 - protein, 7 aa, Mus musculus
SIPSVAR

SEQ ID NO: 565 - protein, 13 aa, Mus musculus
GTEAIIIPYALHR

SEQ ID NO: 566 - protein, 7 aa, Mus musculus
LSQEDIR

SEQ ID NO: 567 - protein, 12 aa, Mus musculus
DELNGFFNTLIR

SEQ ID NO: 568 - protein, 9 aa, Mus musculus
LTILQVLHK

SEQ ID NO: 569 - protein, 7 aa, Mus musculus
LGLLVVK

SEQ ID NO: 570 - protein, 7 aa, Mus musculus
DAFNIQR

SEQ ID NO: 571 - protein, 9 aa, Mus musculus
ALDFQSYAK

SEQ ID NO: 572 - protein, 9 aa, Mus musculus
VVQEDYVLK

SEQ ID NO: 573 - protein, 11 aa, Mus musculus
AGFLSLFGYTK

SEQ ID NO: 574 - protein, 9 aa, Mus musculus
FVYSLLGPR

SEQ ID NO: 575 - protein, 10 aa, Mus musculus
EWVEVSQLQR

SEQ ID NO: 576 - protein, 12 aa, Mus musculus
VFGYQSVDGDHR

SEQ ID NO: 577 - protein, 10 aa, Mus musculus
VPPLIASFVR

SEQ ID NO: 578 - protein, 14 aa, Mus musculus
GSAPPGPVPEGQIR

SEQ ID NO: 579 - protein, 8 aa, Mus musculus
ALWSSSEK

SEQ ID NO: 580 - protein, 9 aa, Mus musculus
TENIETIDR

SEQ ID NO: 581 - protein, 10 aa, Mus musculus
APFFEYNIHK

SEQ ID NO: 582 - protein, 12 aa, Mus musculus
ELSEEDVDAVVR

SEQ ID NO: 583 - protein, 8 aa, Mus musculus
DEYLLNFK

SEQ ID NO: 584 - protein, 13 aa, Mus musculus
IFSSHIPYYLVPK

SEQ ID NO: 585 - protein, 8 aa, Mus musculus
ANYEHIIK

SEQ ID NO: 586 - protein, 9 aa, Mus musculus
GLALPQDFR

SEQ ID NO: 587 - protein, 10 aa, Mus musculus
DSSDVYSTQK

SEQ ID NO: 588 - protein, 12 aa, Mus musculus
GASAAGSALVYR

SEQ ID NO: 589 - protein, 13 aa, Mus musculus
QLEFLQPLVEDPR

SEQ ID NO: 590 - protein, 7 aa, Mus musculus
IVAFAGK

SEQ ID NO: 591 - protein, 8 aa, Mus musculus
DAASLTNR

SEQ ID NO: 592 - protein, 8 aa, Mus musculus
LSAELGLR

SEQ ID NO: 593 - protein, 7 aa, Mus musculus
TVFFQQR

SEQ ID NO: 594 - protein, 8 aa, Mus musculus
SISLLEER

SEQ ID NO: 595 - protein, 7 aa, Mus musculus
DLVHSLK

SEQ ID NO: 596 - protein, 11 aa, Mus musculus
TAHLHVLAVHR

SEQ ID NO: 597 - protein, 9 aa, Mus musculus
GAAVALNIR

SEQ ID NO: 598 - protein, 13 aa, Mus musculus
YVTLIYTNYENGK

SEQ ID NO: 599 - protein, 7 aa, Mus musculus
GLTHSIR

SEQ ID NO: 600 - protein, 9 aa, Mus musculus
AILSYLAAK

SEQ ID NO: 601 - protein, 7 aa, Mus musculus
LYYFNGR

SEQ ID NO: 602 - protein, 10 aa, Mus musculus
EESYDLILSR

SEQ ID NO: 603 - protein, 7 aa, Mus musculus
YFPVFEK

SEQ ID NO: 604 - protein, 9 aa, Mus musculus
AILNYIASK

SEQ ID NO: 605 - protein, 7 aa, Mus musculus
YFPAFEK

SEQ ID NO: 606 - protein, 9 aa, Mus musculus
IAAFLQSDR

SEQ ID NO: 607 - protein, 8 aa, Mus musculus
QFSLFLGK

SEQ ID NO: 608 - protein, 8 aa, Mus musculus
FTWFAGEK

SEQ ID NO: 609 - protein, 7 aa, Mus musculus
SQWLDVK

SEQ ID NO: 610 - protein, 9 aa, Mus musculus
NEDLINGIK

SEQ ID NO: 611 - protein, 8 aa, Mus musculus
NHFPEALR

SEQ ID NO: 612 - protein, 8 aa, Mus musculus
SFSEFVEK

SEQ ID NO: 613 - protein, 7 aa, Mus musculus
EDAIFNR

SEQ ID NO: 614 - protein, 9 aa, Mus musculus
DDVDLVEFK

SEQ ID NO: 615 - protein, 12 aa, Mus musculus
YVNEEEIEDVLK

SEQ ID NO: 616 - protein, 10 aa, Mus musculus
DQPQVPAIFR

SEQ ID NO: 617 - protein, 10 aa, Mus musculus
DYIVDAGFGR

SEQ ID NO: 618 - protein, 9 aa, Mus musculus
DNVDLVEFK

SEQ ID NO: 619 - protein, 12 aa, Mus musculus
SLTEEEIEDVLR

SEQ ID NO: 620 - protein, 7 aa, Mus musculus
IGYQSTR

SEQ ID NO: 621 - protein, 9 aa, Mus musculus
EEEIEEVLK

SEQ ID NO: 622 - protein, 9 aa, Mus musculus
DNIDLVEFK

SEQ ID NO: 623 - protein, 12 aa, Mus musculus
DQPQVPAIFHLR

SEQ ID NO: 624 - protein, 10 aa, Mus musculus
SVFGIHLETK

SEQ ID NO: 625 - protein, 11 aa, Mus musculus
DVVVSYYNFYK

SEQ ID NO: 626 - protein, 11 aa, Mus musculus
VSYGSWYQHVK

SEQ ID NO: 627 - protein, 8 aa, Mus musculus
TYAFPYTK

SEQ ID NO: 628 - protein, 13 aa, Mus musculus
EEYQQEILSDIEK

SEQ ID NO: 629 - protein, 8 aa, Mus musculus
GEDFFTLK

SEQ ID NO: 630 - protein, 9 aa, Mus musculus
NTGVYLISR

SEQ ID NO: 631 - protein, 12 aa, Mus musculus
DIAPSNHYSLQR

SEQ ID NO: 632 - protein, 10 aa, Mus musculus
TTYWIDYILR

SEQ ID NO: 633 - protein, 10 aa, Mus musculus
IGVSFLVLPK

SEQ ID NO: 634 - protein, 7 aa, Mus musculus
YNLLPAK

SEQ ID NO: 635 - protein, 9 aa, Mus musculus
YLSEDIANK

SEQ ID NO: 636 - protein, 9 aa, Mus musculus
TLASALHER

SEQ ID NO: 637 - protein, 7 aa, Mus musculus
LAGALGR

SEQ ID NO: 638 - protein, 12 aa, Mus musculus
GHHTVLLLSEGR

SEQ ID NO: 639 - protein, 11 aa, Mus musculus
YFSLPSVVFSR

SEQ ID NO: 640 - protein, 8 aa, Mus musculus
IFIDAQWK

SEQ ID NO: 641 - protein, 13 aa, Mus musculus
TYAVSHTQEDLNR

SEQ ID NO: 642 - protein, 9 aa, Mus musculus
DISEEVLNK

SEQ ID NO: 643 - protein, 11 aa, Mus musculus
VLWGSWYDHVK

SEQ ID NO: 644 - protein, 12 aa, Mus musculus
VFDVAVLAPLLK

SEQ ID NO: 645 - protein, 9 aa, Mus musculus
TALTFQQIK

SEQ ID NO: 646 - protein, 10 aa, Mus musculus
YEDLVGDPVK

SEQ ID NO: 647 - protein, 7 aa, Mus musculus
DPALDLK

SEQ ID NO: 648 - protein, 8 aa, Mus musculus
VNSPEEVK

SEQ ID NO: 649 - protein, 14 aa, Mus musculus
LYPGDAVSLQGAAR

SEQ ID NO: 650 - protein, 7 aa, Mus musculus
ESLQVVR

SEQ ID NO: 651 - protein, 13 aa, Mus musculus
IASGLGLAWIIGR

SEQ ID NO: 652 - protein, 9 aa, Mus musculus
NLDEDVVDK

SEQ ID NO: 653 - protein, 9 aa, Mus musculus
IVLETSFEK

SEQ ID NO: 654 - protein, 10 aa, Mus musculus
NHFTVAQNER

SEQ ID NO: 655 - protein, 11 aa, Mus musculus
VSWGSWFDHVK

SEQ ID NO: 656 - protein, 11 aa, Mus musculus
LELLAYLLGEK

SEQ ID NO: 657 - protein, 11 aa, Mus musculus
QFFQVPLNIPK

SEQ ID NO: 658 - protein, 7 aa, Mus musculus
ISTQQVK

SEQ ID NO: 659 - protein, 8 aa, Mus musculus
YQHLWNIK

SEQ ID NO: 660 - protein, 9 aa, Mus musculus
ADVGLVEDK

SEQ ID NO: 661 - protein, 12 aa, Mus musculus
LLDLGVLVPLLR

SEQ ID NO: 662 - protein, 11 aa, Mus musculus
LYAQPGDPGER

SEQ ID NO: 663 - protein, 9 aa, Mus musculus
EGETPLETK

SEQ ID NO: 664 - protein, 9 aa, Mus musculus
SLAEGNPDR

SEQ ID NO: 665 - protein, 7 aa, Mus musculus
EQGAEVR

SEQ ID NO: 666 - protein, 7 aa, Mus musculus
DPGLNLK

SEQ ID NO: 667 - protein, 7 aa, Mus musculus
ALPSAPR

SEQ ID NO: 668 - protein, 7 aa, Mus musculus
LSQEQVR

SEQ ID NO: 669 - protein, 7 aa, Mus musculus
ESIQVLR

SEQ ID NO: 670 - protein, 7 aa, Mus musculus
EAVHAWR

SEQ ID NO: 671 - protein, 10 aa, Mus musculus
QHIYVHATWR

SEQ ID NO: 672 - protein, 10 aa, Mus musculus
NPAISLVEER

SEQ ID NO: 673 - protein, 8 aa, Mus musculus
VPEVNDLR

SEQ ID NO: 674 - protein, 8 aa, Mus musculus
NSAATAEK

SEQ ID NO: 675 - protein, 7 aa, Mus musculus
ALVEDPR

SEQ ID NO: 676 - protein, 7 aa, Mus musculus
GDPTLGK

SEQ ID NO: 677 - protein, 10 aa, Mus musculus
DLSLDLLLPR

SEQ ID NO: 678 - protein, 10 aa, Mus musculus
DALSVSQAWR

SEQ ID NO: 679 - protein, 12 aa, Mus musculus
QVPSSPAGLGER

SEQ ID NO: 680 - protein, 7 aa, Mus musculus
DPLTVIR

SEQ ID NO: 681 - protein, 9 aa, Mus musculus
SVHSELEQR

SEQ ID NO: 682 - protein, 7 aa, Mus musculus
HTLPFAK

SEQ ID NO: 683 - protein, 7 aa, Mus musculus
WASSTEK

SEQ ID NO: 684 - protein, 11 aa, Mus musculus
EPILAAQAVPR

SEQ ID NO: 685 - protein, 9 aa, Mus musculus
QPELSLSPR

SEQ ID NO: 686 - protein, 7 aa, Mus musculus
QPELTLR

SEQ ID NO: 687 - protein, 12 aa, Mus musculus
AIQTLPEALQQR

SEQ ID NO: 688 - protein, 8 aa, Mus musculus
APLAQTTR

SEQ ID NO: 689 - protein, 8 aa, Mus musculus
SHGFVVLK

SEQ ID NO: 690 - protein, 7 aa, Mus musculus
SQVDIVK

SEQ ID NO: 691 - protein, 7 aa, Mus musculus
WSLPYEK

SEQ ID NO: 692 - protein, 11 aa, Mus musculus
VITSGFNALEK

SEQ ID NO: 693 - protein, 8 aa, Mus musculus
ILQSTAGK

SEQ ID NO: 694 - protein, 13 aa, Mus musculus
GIDYEIVPINLIK

SEQ ID NO: 695 - protein, 14 aa, Mus musculus
VDLSPYPTISHINK

SEQ ID NO: 696 - protein, 11 aa, Homo sapiens
VALTLDLDLEK

SEQ ID NO: 697 - protein, 8 aa, Homo sapiens
QELIAWEK

(19) United States
(12) Patent Application Publication
(10) Pub. No.: US 2011/0177491 A1
(43) Pub. Date: Jul. 21, 2011

(54) PEPTIDE FOR USE IN SIMULTANEOUS PROTEIN QUANTIFICATION OF METABOLIZING ENZYMES USING MASS SPECTROMETRIC ANALYSIS APPARATUS

(75) Inventors: Junichi Kamiie, Miyagi (JP); Sumio Ohtsuki, Miyagi (JP); Tetsuya Terasaki, Miyagi (JP)

(73) Assignee: TOHOKU UNIVERSITY, Sendai-shi, Miyagi (JP)

(21) Appl. No.: 13/120,705

(22) PCT Filed: Sep. 28, 2009
(86) PCT No.: PCT/JP2009/004948

§ 371 Date: Mar. 24, 2011

(30) Foreign Application Priority Data
Sep. 29, 2008 (JP) 2008-251212

(51) Int. Cl. C12Q 1/00; C12N 9/00

(52) U.S. Cl. 435/4; 435/183

(57) ABSTRACT

There are provided a peptide consisting of an amino acid sequence for simultaneously quantifying absolute amounts of metabolizing enzyme proteins in a biological sample at high sensitivity and a method for using the same. A peptide which can be detected at high sensitivity with a mass spectrometer that enables highly sensitive simultaneous quantification of metabolizing enzymes, intracellular proteins, is selected, and the amino acid sequence thereof is identified. Using a stable-isotope-labeled peptide having the same amino acid sequence as the amino acid sequence of this peptide to be quantified, a mass spectrometry by LC-MS/MS is performed at predetermined concentration levels of the stable-isotope-labeled peptide to create a calibration curve. The stable-isotope-labeled peptide is added to a peptide fragment obtained by fragmenting metabolizing enzyme proteins to be quantified in a sample with trypsin, amass spectrometry by LC-MS/MS is performed to calculate amass spectrum area ratio of the metabolizing enzyme protein peptides to be quantified and the stable-isotope-labeled peptide, and a quantitative value is obtained from the area ratio using the calibration curve.

### TECHNICAL FIELD

The present invention relates to a peptide for use in simultaneous quantification of metabolizing enzyme proteins in humans or other mammals with a mass spectrometer and a method of using the same. More specifically, the present invention relates to a peptide consisting of a partial amino acid sequence of a human metabolizing enzyme protein or the like for use in simultaneous quantification of absolute amounts of two or more of human or mammal metabolizing enzyme proteins in a biological sample with amass spectrometer at high sensitivity and a method of using the same.

### BACKGROUND ART

Metabolizing enzymes occurring in mammals including humans include drug metabolizing enzymes, steroid metabolizing enzymes, amino acid metabolizing enzymes, and nucleic acid metabolizing enzymes. Among these metabolizing enzymes, drug metabolizing enzymes are important enzymes that are involved in effectiveness and occurrence of toxicities of a drug by modifying the drug by metabolism. Examples of such metabolizing enzymes include CYP (P-450), glucuronic acid conjugating enzymes, sulfuric acid conjugating enzymes, and glucuronide conjugating enzymes. These enzyme molecules are subject to induction and inhibition of expressions thereof due to various factors, and such variations in the expressions result in varied effectiveness and toxicities of a drug. As expression profiles of drug-metabolizing enzymes thus determine how a drug is metabolized, these expression profiles are very important information in development of a drug.

An expression profile of a drug metabolizing enzyme can be analyzed at an mRNA level by utilizing PCR, DNA chips, and the like. However, mRNA expression is not necessarily consistent with expression of a protein, which is actual manifestation of an activity of the enzyme. Furthermore, one drug is often metabolized by more than one metabolizing enzyme. In such a case, the magnitude of contribution of each metabolizing enzyme cannot be analyzed by mRNA expression. Achievement of comparison between different metabolizing enzymes by analyzing metabolizing enzymes with a protein expression level and further obtaining an absolute expression level has been awaited.

One example of methods for detecting a metabolizing enzyme protein is a method using an antibody. Although a protein can be detected and quantified by Western blot in this method, it is not only very difficult to prepare a specific antibody, but also it requires considerable time and labor to construct profiles of two or more molecules.

Meanwhile, mass spectrometry techniques have been dramatically advanced, investigated, and utilized to detect and measure various biomaterials in recent years. Mass spectrometers having various functions have been developed, including a mass spectrometer in use for electrosprayionization (ESI), a mass spectrometer in use for liquid chromatography-mass spectrometry (LC-MS), and an MS/MS or tandem mass (tandem MS) spectrometer, which consists of two connected mass spectrometers. Mass spectrometers having these functions in combination are used in detection, measurement, or quantification of biomaterials (Patent Documents 1 to 3).

The inventors of the present invention have invented a method for simultaneous quantification of absolute expression levels of 20 or more membrane proteins with amass spectrometer (Patent Document 4). This invention is a method for obtaining an absolute expression level of a target protein by selecting a highly sensitive peptide from peptides obtained by digesting the target protein with trypsin and using a stable-isotope-labeled peptide that has the same amino acid sequence as the amino acid sequence of the selected peptide to quantify an absolute expression level of a trypsin-digested peptide to be quantified in a biological sample. It is therefore critical to select a highly sensitive peptide in a target protein to achieve quantification at high sensitivity, precision, and reliability.

However, the above-mentioned invention described in Patent Document 4 is an invention related to a method for quantifying an absolute expression level of a cell membrane protein and an amino acid sequence of a peptide of the cell membrane protein, and information on a peptide that can be used for quantification of a metabolizing enzyme, an intracellular protein, was unknown. As an enormous amount of proteins exist in the cell as compared with the cell membrane, quantification of a metabolizing enzyme, an intracellular protein, involves quantification of trace amounts of proteins in a more complicated protein sample, and it was anticipated to be more difficult to select a peptide to be quantified for quantification of a metabolizing enzyme in an intracellular protein sample as compared with selection of a peptide to be quantified in a cell membrane protein.

### CITATION LIST

### Patent Document

Patent Document 1: Japanese Patent Laid-Open No. 2004-28993
Patent Document 2: Japanese Patent Laid-Open No. 2004-77276
Patent Document 3: National Publication of International Patent Application No. 2004-533610
Patent Document 4: International Publication No. WO 07/055,116

### DISCLOSURE OF THE INVENTION

### Problems to be Solved by the Invention

An object of the present invention is to provide a peptide consisting of an amino acid sequence that can simultaneously quantify absolute protein amounts of metabolizing enzymes in a biological sample at high sensitivity and a method of using the same.

### Means for Solving the Problems

The inventors of the present invention conducted various researches to achieve the foregoing object and found that the presence of a specific amino acid suppresses ionization of peptides and lowers sensitivity, and that the presence of a different specific amino acid increases efficiency of collision-induced dissociation and facilitates ionization of peptides, resulting in high sensitivity. The inventors of the present invention selected a peptide that can be detected with amass spectrometer at high sensitivity and can simultaneously quantify metabolizing enzymes, intracellular proteins, at high sensitivity, identified an amino acid sequence of the peptide by scoring peptides according to criteria and narrowing down a candidate from two or more peptides by total scores, and thus accomplished the present invention.

Specifically, the present invention relates to (1) a peptide for use in simultaneous protein quantification of metabolizing enzymes with a mass spectrometer, consisting of the partial amino acid sequence of a human metabolizing enzyme protein set forth in any one of SEQ ID NOS: 1 to 412, (2) a peptide for use in simultaneous protein quantification of metabolizing enzymes with a mass spectrometer, consisting of the partial amino acid sequence of a mouse metabolizing enzyme protein set forth in any one of SEQ ID NOS: 413 to 695, (3) a peptide for use in simultaneous protein quantification of metabolizing enzymes with a mass spectrometer, consisting of an amino acid sequence including deletion, substitution, or addition of one or two amino acids in the amino acid sequence set forth in any one of SEQ ID NOS: 1 to 695, and (4) a stable-isotope-labeled peptide, wherein one or more of peptide-constituting amino acids contain any one or more of ¹⁵N, ¹³C, ¹⁸O, and ²H in the peptide according to any one of the above (1) to (3).

Furthermore, the present invention relates to (5) a kit for simultaneous protein quantification of metabolizing enzymes, comprising the peptide according to any one of the above (1) to (3) and the stable-isotope-labeled peptide according to the above (4), and (6) a method of using the peptide according to any one of the above (1) to (3) and the stable-isotope-labeled peptide according to the above (4) as a probe for simultaneous protein quantification of metabolizing enzymes.

Furthermore, the present invention relates to (7) a method for simultaneous protein quantification of metabolizing enzymes with a liquid chromatograph-tandem mass spectrometer (LC-MS/MS) using a stable-isotope-labeled peptide, comprising the steps of: (a) performing a mass spectrometry by liquid chromatograph-tandem mass spectrometer (LC-MS/MS) using the peptide according to any one of the above (1) to (3) and the stable-isotope-labeled peptide according to the above (4) at predetermined concentration levels of these peptides to create a calibration curve; (b) performing a mass spectrometry by LC-MS/MS by adding the stable-isotope-labeled peptide according to the above (4) to peptide fragments obtained by fragmenting metabolizing enzyme proteins to be quantified in a sample with trypsin to calculate a mass spectrum area ratio of a metabolizing enzyme protein peptide to be quantified and the stable-isotope-labeled peptide; and (c) obtaining a quantitative value from the area ratio using the calibration curve.

### Advantageous Effects of Invention

Quantification of metabolizing enzyme proteins, which was difficult by conventional methods, can be achieved conveniently and rapidly at high precision by a method for quantification of metabolizing enzyme proteins by mass spectrometry using a peptide consisting of the amino acid sequence of the present invention. Furthermore, as metabolizing enzyme proteins can be quantified by the method for quantification of metabolizing enzyme proteins of the present invention without using an antibody, the step of preparing an antibody, which conventionally required the longest time, can be omitted, and further metabolizing enzymes for which an antibody cannot be prepared can be quantified. Therefore, a method of wide application for highly precise quantification of metabolizing enzyme proteins, intracellular proteins, can be provided. Accordingly, a method for quantification of metabolizing enzyme proteins utilizing the present invention can be expected to contribute greatly to understanding of drug interactions, individual variations, and toxicities. Particularly in development of a novel drug, information on metabolizing enzymes of which expressions are changed by a novel drug candidate substance and the extents to which these expressions are changed has a very important significance to predict interactions and toxicities of the substance. The method of the present invention can therefore be expected to contribute greatly to promotion of development of a novel drug.

### BRIEF DESCRIPTION OF DRAWINGS

FIG. 1 A diagram showing results of measurement by nanoLC-MS/MS by adding 50 fmol each of ¹³C₆,¹⁵N-labeled peptides to 1 fmol of non-labeled peptides to be quantified. 1 fmol of selected peptides (CYP) can be detected by nanoLC.

FIG. 2 An example of analysis of absolute expression levels of CYP from the human liver: a peak of a peptide to be quantified (left panel: in red, marked with *) and a peak of a labeled peptide as an internal standard (right panel: in red, marked with *) in the analysis of the human liver. Both the peaks were detected at the same elution time. In the graphs, the horizontal axis represents elution time (min), and the vertical axis represents intensity (cps).

FIG. 3 An example of analysis of absolute expression levels of CYP from the human liver: a peak of a peptide to be quantified (left panel: in red, marked with *) and a peak of a labeled peptide as an internal standard (right panel: in red, marked with *) in the analysis of the human liver. Both the peaks were detected at the same elution time. In the graphs, the horizontal axis represents elution time (min), and the vertical axis represents intensity (cps).

FIG. 4 An example of analysis of absolute expression levels of CYP from the human liver: a peak of a peptide to be quantified (left panel: in red, marked with *) and a peak of a labeled peptide as an internal standard (right panel: in red, marked with *) in the analysis of the human liver. Both the peaks were detected at the same elution time. In the graphs, the horizontal axis represents elution time (min), and the vertical axis represents intensity (cps).

FIG. 5 An example of analysis of absolute expression levels of CYP from the human liver: The calibration curve of each CYP molecule is shown. The horizontal axis represents the amount of a peptide to be quantified (fmol), and the vertical axis represents the peak area ratio of the peptide to be quantified and the labeled peptide. In the graphs, the blue circle represents a reference standard of a synthesized peptide, and the green triangle represents a peptide to be quantified in a sample.

FIG. 6 An example of analysis of absolute expression levels of CYP from the human liver: The calibration curve of each CYP molecule is shown. The horizontal axis represents the amount of a peptide to be quantified (fmol), and the vertical axis represents the peak area ratio of the peptide to be quantified and the labeled peptide. In the graphs, the blue circle represents a reference standard of a synthesized peptide, and the green triangle represents a peptide to be quantified in a sample.

FIG. 7 A diagram showing results of measurement by conventional LC-MS/MS by adding 500 fmol each of ¹³C₆,¹⁵N-labeled peptides to 10 fmol of non-labeled peptides to be quantified. Conventional HPLC can detect 10 fmol of peptides (Ara-C metabolizing enzymes).

FIG. 8 An example of analysis of absolute expression levels of Ara-C-related metabolizing enzymes from human leukemic cells: a peak of a peptide to be quantified (left panel: in red, marked with *) and a peak of a labeled peptide as an internal standard (right panel: in red, marked with *) in the analysis of human leukemic cells. Both the peaks were detected at the same elution time. In the graphs, the horizontal axis represents elution time (min), and the vertical axis represents intensity (cps).

FIG. 9 An example of analysis of absolute expression levels of Ara-C-related metabolizing enzymes from human leukemic cells: a peak of a peptide to be quantified (left panel: in red, marked with *) and a peak of a labeled peptide as an internal standard (right panel: in red, marked with *) in the analysis of human leukemic cells. Both the peaks were detected at the same elution time. In the graphs, the horizontal axis represents elution time (min), and the vertical axis represents intensity (cps).

FIG. 10 An example of analysis of expression levels of Ara-C-related metabolizing enzymes from human leukemic cells. The calibration curve of each Ara-C metabolizing enzyme molecule is shown. The horizontal axis represents the amount of a peptide to be quantified (fmol), and the vertical axis represents the peak area ratio of the peptide to be quantified and the labeled peptide. In the graphs, the blue circle represents a reference standard of a synthesized peptide, and the green triangle represents a peptide to be quantified in a sample.

FIG. 11 An example of analysis of expression levels of Ara-C-related metabolizing enzymes from human leukemic cells. The calibration curve of each Ara-C metabolizing enzyme molecule is shown. The horizontal axis represents the amount of a peptide to be quantified (fmol), and the vertical axis represents the peak area ratio of the peptide to be quantified and the labeled peptide. In the graph, the blue circle represents a reference standard of a synthesized peptide, and the green triangle represents a peptide to be quantified in a sample.

### MODE OF CARRYING OUT THE INVENTION

### (Peptides Consisting of Partial Amino Acid Sequence of Human Metabolizing Enzyme Protein)

The peptide of the present invention is a peptide for use in simultaneous protein quantification of metabolizing enzymes with a mass spectrometer, consisting of a partial amino acid sequence of a human metabolizing enzyme protein. Specific examples of the human metabolizing enzyme protein can include CYP1A1 (SwissProt accession number: P04798), CYP1A2 (SwissProt accession number: P05177), CYP1B1 (SwissProt accession number: Q16678), CYP2A6 (SwissProt accession number: P11509), CYP2A7 (SwissProt accession number: P20853), CYP2A13 (SwissProt accession number: Q16696), CYP2B6 (SwissProt accession number: P20813), CYP2C8 (SwissProt accession number: P10632), CYP2C9 (SwissProt accession number: P11712), CYP2C18 (SwissProt accession number: P33260), CYP2C19 (SwissProt accession number: P33261), CYP2D6 (SwissProt accession number: P10635), CYP2E1 (SwissProt accession number: P05181), CYP2F1 (SwissProt accession number: P24903), CYP2J2 (SwissProt accession number: P51589), CYP2R1 (SwissProt accession number: Q6VVX0), CYP2S1 (SwissProt accession number: Q96SQ9), CYP2W1 (SwissProt accession number: Q8TAV3), CYP3A4 (SwissProt accession number: P08684), CYP3A5 (SwissProt accession number: P20815), CYP3A7 (SwissProt accession number: P24462), CYP3A43 (SwissProt accession number: Q9HB55), CYP4A11 (SwissProt accession number: Q02928), CYP4B1 (SwissProt accession number: P13584), CYP4F2 (SwissProt accession number: P78329), CYP4F3 (SwissProt accession number: Q08477), CYP4F8 (SwissProt accession number: P98187), CYP4F11 (SwissProt accession number: Q9HBI6), CYP4F12 (SwissProt accession number: Q9HCS2), CYP4F22 (SwissProt accession number: Q6NT55), CYP4V2 (SwissProt accession number: Q6ZWL3), CYP4X1 (SwissProt accession number: Q8N118), CYP4Z1 (SwissProt accession number: Q86W10), CYP7A1 (SwissProt accession number: P22680), CYP7B1 (SwissProt accession number: 075881), CYP8B1 (SwissProt accession number: Q9UNU6), CYP11B1 (SwissProt accession number: P15538), CYP11B2 (SwissProt accession number: P19099), CYP17A1 (SwissProt accession number: P05093), CYP19A1 (SwissProt accession number: P11511), CYP21A2 (SwissProt accession number: P08686), CYP24A1 (SwissProt accession number: Q07973), CYP26A1 (SwissProt accession number: 043174), CYP26B1 (SwissProt accession number: Q9NR63), CYP26C1 (SwissProt accession number: Q6VOLO), CYP27A1 (SwissProt accession number: Q02318), CYP27B1 (SwissProt accession number: 015528), CYP27C1 (SwissProt accession number: Q4GOS4), CYP39A1 (SwissProt accession number: Q9NYL5), CYP46A1 (SwissProt accession number: Q9Y6A2), CYP51A1 (SwissProt accession number: Q16850), UGT1A1 (SwissProt accession number: P22309), UGT1A3 (SwissProt accession number: P35503), UGT1A4 (SwissProt accession number: P22310), UGT1A5 (SwissProt accession number: P35504), UGT1A6 (SwissProt accession number: P19224), UGT1A7 (SwissProt accession number: Q9HAW7), UGT1A10 (SwissProt accession number: Q9HAW8), UGT2A1 (SwissProt accession number: Q9Y4X1), UGT2B4 (SwissProt accession number: P06133), UGT2B7 (SwissProt accession number: P16662), UGT2B10 (SwissProt accession number: P36537), UGT2B11 (SwissProt accession number: 075310), UGT2B17 (SwissProt accession number: 075795), UGT2B28 (SwissProt accession number: Q9BY64), GSTA1 (SwissProt accession number: P08263), GSTA2 (SwissProt accession number: P09210), GSTA3 (SwissProt accession number: Q16772), GSTA4 (SwissProt accession number: 015217), GSTA5 (SwissProt accession number: Q7RTV2), GSTK1 (SwissProt accession number: Q9Y2Q3), GSTM1 (SwissProt accession number: P09488), GSTM2 (SwissProt accession number: P28161), GSTM3 (SwissProt accession number: P21266), GSTM4 (SwissProt accession number: Q03013), GSTM5 (SwissProt accession number: P46439), GSTP1 (SwissProt accession number: P09211), GSTT1 (SwissProt accession number: P30711), GSTT2 (SwissProt accession number: P30712), MGST1 (SwissProt accession number: P10620), MGST2 (SwissProt accession number: Q99735), MGST3 (SwissProt accession number: 014880), SULT1A2 (SwissProt accession number: P50226), SULT1A3 (SwissProt accession number: P50224), SULT1B1 (SwissProt accession number: 043704), SULT1C2 (SwissProt accession number: P000338), SULT1C3 (SwissProt accession number: Q6IMI6), SULT1C4 (SwissProt accession number: 075897), SULT1E1 (SwissProt accession number: P49888), SULT2A1 (SwissProt accession number: Q06520), SULT2B1 (SwissProt accession number: 000204), SULT4A1 (SwissProt accession number: Q9BR01), SULT4S6 (SwissProt accession number: Q7LFX5), P450R (SwissProt accession number: P16435), MGMT (SwissProt accession number: P16455), dCK (SwissProt accession number: P27707), CDA (SwissProt accession number: P32320), cN-IA (SwissProt accession number: Q9BX13), cN-IB (SwissProt accession number: Q96P26), cN-II (SwissProt accession number: P49902), cN-III (SwissProt accession number: Q9H0P0), dNT-1 (SwissProt accession number: Q8TCD5), dNT-2 (SwissProt accession number: Q9NPB1), Ecto-5′-NT (SwissProt accession number: P21589), RRM1 (SwissProt accession number: P23921), RRM2 (SwissProt accession number: P31350), UMP/CMPK (SwissProt accession number: P30085), dCMPDA (SwissProt accession number: P32321), CTP synthase 1 (SwissProt accession number: P17812), and CTP synthase 2 (SwissProt accession number: Q9NRF8).

The peptide of the present invention is a peptide consisting of any of partial amino acid sequences of the above-mentioned human metabolizing enzyme proteins. Specific examples of the peptide include peptides each consisting of any of amino acid sequences of SEQ ID NOS: 1 to 5, which are partial sequences of CYP1A1; amino acid sequences of SEQ ID NOS: 6 to 8, which are partial sequences of CYP1A2; and amino acid sequences of SEQ ID NOS: 9 to 14, which are partial sequences of CYP1B1. CYP1A1, CYP1A2, and CYP1B1 can be quantified using any of the corresponding peptides.

Similarly, peptides each consisting of any of amino acid sequences of SEQ ID NOS: 15 to 17, which are partial sequences of CYP2A6; amino acid sequences of SEQ ID NOS: 18 and 19, which are partial sequences of CYP2A7; amino acid sequences of SEQ ID NOS: 20 and 21, which are partial sequences of CYP2A13; amino acid sequences of SEQ ID NOS: 22 to 25, which are partial sequences of CYP2B6; amino acid sequences of SEQ ID NOS: 26 to 29, which are partial sequences of CYP2C8; amino acid sequences of SEQ ID NOS: 30 and 31, which are partial sequences of CYP2C9; amino acid sequences of SEQ ID NOS: 32 to 34, which are partial sequences of CYP2C18; amino acid sequences of SEQ ID NOS: 35 to 37, which are partial sequences of CYP2C19; amino acid sequences of SEQ ID NOS: 38 and 39, which are partial sequences of CYP2D6; amino acid sequences of SEQ ID NOS: 40 to 46, which are partial sequences of CYP2E1; an amino acid sequence of SEQ ID NO: 47, which is a partial sequence of CYP2F1; amino acid sequences of SEQ ID NOS: 48 to 55, which are partial sequences of CYP2J2; amino acid sequences of SEQ ID NOS: 56 to 63, which are partial sequences of CYP2R1; amino acid sequences of SEQ ID NOS: 64 to 72, which are partial sequences of CYP2S1; and amino acid sequences of SEQ ID NOS: 73 to 79, which are partial sequences of CYP2W1, are also the peptides of the present invention. CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, and CYP2W1 can be quantified using any of the corresponding peptides.

Similarly, peptides each consisting of any of amino acid sequences of SEQ ID NOS: 80 and 81, which are partial sequences of CYP3A4; amino acid sequences of SEQ ID NOS: 82 to 84, which are partial sequences of CYP3A5; amino acid sequences of SEQ ID NOS: 85 to 88, which are partial sequences of CYP3A7; and amino acid sequences of SEQ ID NOS: 89 to 96, which are partial sequences of CYP3A43, are also the peptides of the present invention. CYP3A4, CYP3A5, CYP3A7, and CYP3A43 can be quantified using any the corresponding peptides.

Similarly, peptides each consisting of any of amino acid sequences of SEQ ID NOS: 97 to 101, which are partial sequences of CYP4A11; amino acid sequences of SEQ ID NOS: 102 to 111, which are partial sequences of CYP4B1; amino acid sequences of SEQ ID NO: 112 to 114, which are partial sequences of CYP4F2; an amino acid sequence of SEQ ID NO: 115, which is a partial sequence of CYP4F3; amino acid sequences of SEQ ID NOS: 116 to 119, which are partial sequences of CYP4F8; amino acid sequences of SEQ ID NOS: 120 to 122, which are partial sequences of CYP4F11; amino acid sequences of SEQ ID NOS: 123 to 127, which are partial sequences of CYP4F12; amino acid sequences of SEQ ID NOS: 128 to 136, which are partial sequences of CYP4F22; amino acid sequences of SEQ ID NOS: 137 to 144, which are partial sequences of CYP4V2; amino acid sequences of SEQ ID NOS: 145 to 154, which are partial sequences of CYP4X1; and amino acid sequences of SEQ ID NOS: 155 to 165, which are partial sequences of CYP4Z1, are also the peptides of the present invention. CYP4A11, CYP4B1, CYP4F2, CYP4F3, CYP4F8, CYP4F11, CYP4F12, CYP4F22, CYP4V2, CYP4X1, and CYP4Z1 can be quantified using any of the corresponding peptides.

Similarly, peptides each consisting of any of amino acid sequences of SEQ ID NOS: 166 to 171, which are partial sequences of CYP7A1; amino acid sequences of SEQ ID NOS: 172 to 178, which are partial sequences of CYP7B1; amino acid sequences of SEQ ID NOS: 179 to 185, which are partial sequences of CYP8B1; amino acid sequences of SEQ ID NOS: 186 to 191, which are partial sequences of CYP11B1; amino acid sequences of SEQ ID NOS: 192 to 195, which are partial sequences of CYP11B2; amino acid sequences of SEQ ID NOS: 196 to 200, which are partial sequences of CYP17A1; and amino acid sequences of SEQ ID NOS: 201 to 207, which are partial sequences of CYP19A1, are also the peptides of the present invention. CYP7A1, CYP7B1, CYP8B1, CYP11B1, CYP11B2, CYP17A1, and CYP19A1 can be quantified using any of the corresponding peptides.

Similarly, peptides each consisting of any of amino acid sequences of SEQ ID NOS: 208 to 210, which are partial sequences of CYP21A2; amino acid sequences of SEQ ID NOS: 211 to 216, which are partial sequences of CYP24A1; amino acid sequences of SEQ ID NOS: 217 to 226, which are partial sequences of CYP26A1; amino acid sequences of SEQ ID NOS: 227 to 238, which are partial sequences of CYP26B1; amino acid sequences of SEQ ID NOS: 239 to 249, which are partial sequences of CYP26C1; amino acid sequences of SEQ ID NOS: 250 to 256, which are partial sequences of CYP27A1; amino acid sequences of SEQ ID NOS: 257 to 265, which are partial sequences of CYP27B1; and amino acid sequences of SEQ ID NOS: 266 to 275, which are partial sequences of CYP27C1, are also the peptides of the present invention. CYP21A2, CYP24A1, CYP26A1, CYP26B1, CYP26C1, CYP27A1, CYP27B1, and CYP27C1 can be quantified using any of the corresponding peptides.

Similarly, peptides each consisting of any of an amino acid sequence of SEQ ID NO: 276, which is a partial sequence of CYP39A1; amino acid sequences of SEQ ID NOS: 277 to 284, which are partial sequences of CYP46A1; and amino acid sequences of SEQ ID NOS: 285 to 296, which are partial sequences of CYP51A1, are also the peptides of the present invention. CYP39A1, CYP46A1, and CYP51A1 can be quantified using any of the corresponding peptides.

Similarly, peptides each consisting of any of an amino acid sequence of SEQ ID NO: 297, which is a partial sequence of UGT1A1; amino acid sequences of SEQ ID NOS: 298 and 299, which are partial sequences of UGT1A3; amino acid sequences of SEQ ID NOS: 300 and 301, which are partial sequences of UGT1A4; an amino acid sequence of SEQ ID NO: 302, which is a partial sequence of UGT1A5; amino acid sequences of SEQ ID NOS: 303 to 305, which are partial sequences of UGT1A6; amino acid sequences of SEQ ID NOS: 306 and 307, which are partial sequences of UGT1A7; and an amino acid sequence of SEQ ID NO: 308, which is a partial sequence of UGT1A10, are also the peptides of the present invention. UGT1A1, UGT1A3, UGT1A4, UGT1A5, UGT1A6, UGT1A7, and UGT1A10 can be quantified using any of the corresponding peptides.

Similarly, peptides each consisting of any of amino acid sequences of SEQ ID NOS: 309 and 310, which are partial sequences of UGT2A1; an amino acid sequence of SEQ ID NO: 311, which is a partial sequence of UGT2B4; amino acid sequences of SEQ ID NOS: 312 and 313, which are partial sequences of UGT2B7; an amino acid sequence of SEQ ID NO: 314, which is a partial sequence of UGT2B10; an amino acid sequence of SEQ ID NO: 315, which is a partial sequence of UGT2B11; an amino acid sequence of SEQ ID NO: 316, which is a partial sequence of UGT2B17; and an amino acid sequence of SEQ ID NO: 317, which is a partial sequence of UGT2B28, are also the peptides of the present invention. UGT2A1, UGT2B4, UGT2B7, UGT2B10, UGT2B11, UGT2B17, and UGT2B28 can be quantified using any of the corresponding peptides.

Similarly, peptides each consisting of any of an amino acid sequence of SEQ ID NO: 318, which is a partial sequence of GSTA1; an amino acid sequence of SEQ ID NO: 319, which is a partial sequence of GSTA2; an amino acid sequence of SEQ ID NO: 320, which is a partial sequence of GSTA3; amino acid sequences of SEQ ID NOS: 321 to 323, which are partial sequences of GSTA4; and an amino acid sequence of SEQ ID NO: 324, which is a partial sequence of GSTA5, are also the peptides of the present invention. GSTA1, GSTA2, GSTA3, GSTA4, and GSTA5 can be quantified using any of the corresponding peptides.

Similarly, peptides each consisting of any of an amino acid sequence of SEQ ID NO: 325, which is a partial sequence of GSTK1; an amino acid sequence of SEQ ID NO: 326, which is a partial sequence of GSTM1; amino acid sequences of SEQ ID NOS: 327 and 328, which are partial sequences of GSTM2; an amino acid sequence of SEQ ID NO: 329, which is a partial sequence of GSTM3; an amino acid sequence of SEQ ID NO: 330, which is a partial sequence of GSTM4; an amino acid sequence of SEQ ID NO: 331, which is a partial sequence of GSTM5; an amino acid sequence of SEQ ID NO: 332, which is a partial sequence of GSTP1; amino acid sequences of SEQ ID NOS: 333 and 334, which are partial sequences of GSTT1; and an amino acid sequence of SEQ ID NO: 335, which is a partial sequence of GSTT2, are also the peptides of the present invention. GSTM1, GSTM2, GSTM3, GSTM4, GSTM5, GSTT1, and GSTT2 can be quantified using any of the corresponding peptides.

Similarly, peptides each consisting of any of an amino acid sequence of SEQ ID NO: 336, which is a partial sequence of MGST1; an amino acid sequence of SEQ ID NO: 337, which is a partial sequence of MGST2; and an amino acid sequence of SEQ ID NO: 338, which is a partial sequence of MGST3, are also the peptides of the present invention. MGST1, MGST2, and MGST3 can be quantified using any of the corresponding peptides.

Similarly, peptides each consisting of any of an amino acid sequence of SEQ ID NO: 339, which is a partial sequence of SULT1A2; an amino acid sequence of SEQ ID NO: 340, which is a partial sequence of SULT1A3; an amino acid sequence of SEQ ID NO: 341, which is a partial sequence of SULT1B1; an amino acid sequence of SEQ ID NO: 342, which is a partial sequence of SULT1C2; an amino acid sequence of SEQ ID NO: 343, which is a partial sequence of SULT1C3; an amino acid sequence of SEQ ID NO: 344, which is a partial sequence of SULT1C4; an amino acid sequence of SEQ ID NO: 345, which is a partial sequence of SULT1E1; an amino acid sequence of SEQ ID NO: 346, which is a partial sequence of SULT2A1; an amino acid sequence of SEQ ID NO: 347, which is a partial sequence of SULT2B1; an amino acid sequence of SEQ ID NO: 348, which is a partial sequence of SULT4A1; and an amino acid sequence of SEQ ID NO: 349, which is a partial sequence of SULT4S6, are also the peptides of the present invention. SULT1A2, SULT1A3, SULT1B1, SULT1C2, SULT1C3, SULT1C4, SULT1E1, SULT2A1, SULT2B1, SULT4A1, and SULT4S6 can be quantified using any of the corresponding peptides.

Similarly, peptides each consisting of any of amino acid sequences of SEQ ID NOS: 350 to 359, which are partial sequences of P450R; amino acid sequences of SEQ ID NOS: 360 and 361, which are partial sequences of MGMT; amino acid sequences of SEQ ID NOS: 362 and 363, which are partial sequences of dCK; and an amino acid sequence of SEQ ID NO: 364, which is a partial sequence of CDA, are also the peptides of the present invention. P450R, MGMT, dCK, and CDA can be quantified using any of the corresponding peptides.

Similarly, peptides each consisting of amino acid sequences of SEQ ID NOS: 365 and 366, which are partial sequences of cN-IA; amino acid sequences of SEQ ID NOS: 367 to 374, which are partial sequences of cN-IB; an amino acid sequence of SEQ ID NO: 375, which is a partial sequence of cN-II; and amino acid sequences of SEQ ID NOS: 376 to 382, which are partial sequences of cN-III, are also the peptides of the present invention. cN-IA, cN-IB, cN-II, and cN-III can be quantified using any of the corresponding peptides.

Similarly, peptides each consisting of any of an amino acid sequence of SEQ ID NO: 383, which is a partial sequence of dNT-1; amino acid sequences of SEQ ID NOS: 384 and 385, which are partial sequences of dNT-2; amino acid sequences of SEQ ID NOS: 386 to 388, which are partial sequences of Ecto-5′-NT; amino acid sequences of SEQ ID NOS: 389 to 392, which are partial sequences of RRM1; amino acid sequences of SEQ ID NOS: 393 and 394, which are partial sequences of RRM2; an amino acid sequence of SEQ ID NO: 395, which is a partial sequence of UMP/CMPK; and an amino acid sequence of SEQ ID NO: 396, which is a partial sequence of dCMPDA, are also the peptides of the present invention. dNT-1, dNT-2, Ecto-5′-NT, RRM1, RRM2, UMP/CMPK, and dCMPDA can be quantified using any of the corresponding peptides.

Similarly, peptides each consisting of any of amino acid sequences of SEQ ID NOS: 397 to 405, which are partial sequences of CTP Synthase 1; and amino acid sequences of SEQ ID NOS: 406 to 412, which are partial sequences of CTP Synthase 2, are also the peptides of the present invention. CTP Synthase 1 and CTP Synthase 2 can be quantified using any of the respective peptides.

### (Peptides Consisting of Partial Amino Acid Sequences of Mouse Metabolizing Enzyme Proteins)

Furthermore, the peptide of the present invention is a peptide consisting of any of partial amino acid sequences of mouse metabolizing enzyme proteins set forth in SEQ ID NOS: 413 to 695. Specific examples of the mouse metabolizing enzyme protein include Cyp11a1 (SwissProt accession number: Q9QZ82), Cyp17a1 (SwissProt accession number: P27786), Cyp19a1 (SwissProt accession number: P28649), Cyp1a1 (SwissProt accession number: P00184), Cyp1a2 (SwissProt accession number: P00186), Cyp21 (SwissProt accession number: P03940), Cyp24a1 (SwissProt accession number: Q64441), Cyp26a1 (SwissProt accession number: 055127), Cyp27a1 (SwissProt accession number: Q9 DBG1), Cyp27b1 (SwissProt accession number: 035084), Cyp2a4 (SwissProt accession number: P15392), Cyp2a5 (SwissProt accession number: P20852), Cyp2b19 (SwissProt accession number: 055071), Cyp2c29 (SwissProt accession number: Q64458), Cyp2c39 (SwissProt accession number: P56656), Cyp2c70 (SwissProt accession number: Q91W64), Cyp2d10 (SwissProt accession number: P24456), Cyp2d26 (SwissProt accession number: Q8CIM7), Cyp2d9 (SwissProt accession number: P11714), Cyp2e1 (SwissProt accession number: Q05421), Cyp2f2 (SwissProt accession number: P33267), Cyp2j5 (SwissProt accession number: 054749), Cyp2r1 (SwissProt accession number: Q6VVW9), Cyp2s1 (SwissProt accession number: Q9 DBX6), Cyp39a1 (SwissProt accession number: Q9JKJ9), Cyp3a11 (SwissProt accession number: Q64459), Cyp3a13 (SwissProt accession number: Q64464), Cyp3a16 (SwissProt accession number: Q64481), Cyp3a25 (SwissProt accession number: 009158), Cyp46a1 (SwissProt accession number: Q9WVK8), Cyp4a14 (SwissProt accession number: 035728), Cyp4b1 (SwissProt accession number: Q64462), Cyp4f14 (SwissProt accession number: Q9EP75), Cyp4v3 (SwissProt accession number: Q9 DBW0), Cyp5a (SwissProt accession number: P36423), Cyp8b1 (SwissProt accession number: 088962), GSTO1 (SwissProt accession number: 009131), ST2B1 (SwissProt accession number: 035400), ST3A1 (SwissProt accession number: 035403), CHST3 (SwissProt accession number: 088199), SNAT (SwissProt accession number: 088816), UD2B5 (SwissProt accession number: P17717), GSTP1 (SwissProt accession number: P19157), GSTM4 (SwissProt accession number: P19639), GSTA4 (SwissProt accession number: P24472), GSTA3 (SwissProt accession number: P30115), GSTM5 (SwissProt accession number: P48774), ST1E1 (SwissProt accession number: P49891), ARY1 (SwissProt accession number: P50294), ARY2 (SwissProt accession number: P50295), ARY3 (SwissProt accession number: P50296), ST1A1 (SwissProt accession number: P52840), UD12 (SwissProt accession number: P70691), CGT (SwissProt accession number: Q64676), UD17C (SwissProt accession number: Q6ZQM8), ST1C1 (SwissProt accession number: Q80VR3), CHST2 (SwissProt accession number: Q80WV3), MGST3 (SwissProt accession number: Q9CPU4), ST1C2 (SwissProt accession number: Q9D939), GSTK1 (SwissProt accession number: Q9DCM2), CHST7 (SwissProt accession number: Q9EP78), CHST1 (SwissProt accession number: Q9EQC0), CHST5 (SwissProt accession number: Q9QUP4), NAT6 (SwissProt accession number: Q9R123), CHST4 (SwissProt accession number: Q9R111), and MAAI (SwissProt accession number: Q9WVL0).

The peptide of the present invention is a peptide consisting of any of partial amino acid sequences of the above-listed proteins. Specific examples of the peptide include peptides each consisting of any of amino acid sequences of SEQ ID NOS: 413 to 422, which are partial sequences of Cyp11a1; amino acid sequences of SEQ ID NOS: 423 to 429, which are partial sequences of Cyp17a1; amino acid sequences of SEQ ID NOS: 430 to 436, which are partial sequences of Cyp19a1; amino acid sequences of SEQ ID NOS: 437 and 438, which are partial sequences of Cyp1a1; and amino acid sequences of SEQ ID NOS: 439 to 443, which are partial sequences of Cyp1a2. Cyp11a1, Cyp17a1, Cyp19a1, Cyp1a1, and Cyp1a2 can be quantified using any of the corresponding peptides.

Similarly, peptides each consisting of any of amino acid sequences of SEQ ID NOS: 444 to 448, which are partial sequences of Cyp21; amino acid sequences of SEQ ID NOS: 449 to 455, which are partial sequences of Cyp24a1; amino acid sequences of SEQ ID NOS: 456 to 464, which are partial sequences of Cyp26a1; amino acid sequences of SEQ ID NOS: 465 to 470, which are partial sequences of Cyp27a1; and amino acid sequences of SEQ ID NOS: 471 to 478, which are partial sequences of Cyp27b1, are also the peptides of the present invention. Cyp21, Cyp24a1, Cyp26a1, Cyp27a1, and Cyp27b1 can be quantified using any of the corresponding peptides.

Similarly, peptides each consisting of any of an amino acid sequence of SEQ ID NO: 479, which is a partial sequence of Cyp2a4; an amino acid sequence of SEQ ID NO: 480, which is a partial sequence of Cyp2a5; amino acid sequences of SEQ ID NOS: 481 to 483, which are partial sequences of Cyp2b19; an amino acid sequence of SEQ ID NO: 484, which is a partial sequence of Cyp2c29; amino acid sequences of SEQ ID NOS: 485 to 488, which are partial sequences of Cyp2c39; amino acid sequences of SEQ ID NOS: 489 to 492, which are partial sequences of Cyp2c70; an amino acid sequence of SEQ ID NO: 493, which is a partial sequence of Cyp2d10; amino acid sequences of SEQ ID NOS: 494 to 497, which are partial sequences of Cyp2d26; amino acid sequences of SEQ ID NOS: 498 and 499, which are partial sequences of Cyp2d9; an amino acid sequence of SEQ ID NO: 500, which is a partial sequence of Cyp2e1; amino acid sequences of SEQ ID NOS: 501 to 505, which are partial sequences of Cyp2f2; amino acid sequences of SEQ ID NOS: 506 to 511, which are partial sequences of Cyp2j5; amino acid sequences of SEQ ID NO: 512 and 513, which are partial sequences of Cyp2r1; and amino acid sequences of SEQ ID NOS: 514 to 526, which are partial sequences of Cyp2s1, are also the peptides of the present invention. Cyp2a4, Cyp2a5, Cyp2b19, Cyp2c29, Cyp2c39, Cyp2c70, Cyp2d10, Cyp2d26, Cyp2d9, Cyp2e1, Cyp2f2, Cyp2j5, Cyp2r1, and Cyp2s1 can be quantified using any of the corresponding peptides.

Similarly, peptides each consisting of any of an amino acid sequence of SEQ ID NO: 527, which is a partial sequence of Cyp39a1; an amino acid sequence of SEQ ID NO: 528, which is a partial sequence of Cyp3a11; amino acid sequences of SEQ ID NOS: 529 to 534, which are partial sequences of Cyp3a13; amino acid sequences of SEQ ID NOS: 535 and 536, which are partial sequences of Cyp3a16; and amino acid sequences of SEQ ID NOS: 537 and 538, which are partial sequences of Cyp3a25, are also the peptides of the present invention. Cyp39a1, Cyp3a11, Cyp3a13, Cyp3a16, and Cyp3a25 can be quantified using any of the corresponding peptides.

Similarly, peptides each consisting of any of amino acid sequences of SEQ ID NOS: 539 to 545, which are partial sequences of Cyp46a1, are also the peptides of the present invention. Cyp46a1 can be quantified using any of the corresponding peptides.

Similarly, peptides each consisting of any of amino acid sequences of SEQ ID NOS: 546 to 550, which are partial sequences of Cyp4a14; amino acid sequences of SEQ ID NOS: 551 to 554, which are partial sequences of Cyp4b1; amino acid sequences of SEQ ID NOS: 555 to 558, which are partial sequences of Cyp4f14; amino acid sequences of SEQ ID NOS: 559 to 566, which are partial sequences of Cyp4v3; amino acid sequences of SEQ ID NOS: 567 to 570, which are partial sequences of Cyp5a; and amino acid sequences of SEQ ID NOS: 571 to 576, which are partial sequences of Cyp8b1, are also the peptides of the present invention. Cyp4a14, Cyp4b1, Cyp4f14, Cyp4v3, Cyp5a, and Cyp8b1 can be quantified using any of the corresponding peptides.

Similarly, peptides each consisting of any of amino acid sequences of SEQ ID NOS: 577 and 578, which are partial sequences of GSTO1; an amino acid sequence of SEQ ID NO: 579, which is a partial sequence of ST2B1; amino acid sequences of SEQ ID NOS: 580 to 585, which are partial sequences of ST3A1; amino acid sequences of SEQ ID NOS: 586 to 595, which are partial sequences of CHST3; an amino acid sequence of SEQ ID NO: 596, which is a partial sequence of SNAT; and an amino acid sequence of SEQ ID NO: 597, which is a partial sequence of UD2B5, are also the peptides of the present invention. GSTO1, ST2B1, ST3A1, CHST3, SNAT, and UD2B5 can be quantified using any of the corresponding peptides.

Similarly, peptides each consisting of any of an amino acid sequence of SEQ ID NO: 598, which is a partial sequence of GSTP1; an amino acid sequence of SEQ ID NO: 599, which is a partial sequence of GSTM4; amino acid sequences of SEQ ID NOS: 600 to 603, which are partial sequences of GSTA4; amino acid sequences of SEQ ID NOS: 604 and 605, which are partial sequences of GSTA3; amino acid sequences of SEQ ID NOS: 606 to 609, which are partial sequences of GSTM5; and amino acid sequences of SEQ ID NOS: 610 to 613, which are partial sequences of ST1E1, are also the peptides of the present invention. GSTP1, GSTM4, GSTA4, GSTA3, GSTM5, and ST1E1 can be quantified using any of the corresponding peptides.

Similarly, peptides each consisting of any of amino acid sequences of SEQ ID NOS: 614 and 615, which are partial sequences of ARY1; amino acid sequences of SEQ ID NOS: 616 to 620, which are partial sequences of ARY2; amino acid sequences of SEQ ID NOS: 621 to 624, which are partial sequences of ARY3; amino acid sequences of SEQ ID NOS: 625 and 626, which are partial sequences of ST1A1; amino acid sequences of SEQ ID NOS: 627 to 629, which are partial sequences of UD12; amino acid sequences of SEQ ID NOS: 630 to 638, which are partial sequences of CGT; amino acid sequences of SEQ ID NOS: 639 to 641, which are partial sequences of UD17C; and amino acid sequences of SEQ ID NOS: 642 and 643, which are partial sequences of ST1C1, are also the peptides of the present invention. ARY1, ARY2, ARY3, ST1A1, UD12, CGT, UD17C, and ST1C1 can be quantified using any of the corresponding peptides.

Similarly, peptides each consisting of any of amino acid sequences of SEQ ID NOS: 644 to 650, which are partial sequences of CHST2; an amino acid sequence of SEQ ID NO: 651, which is a partial sequence of MGST3; amino acid sequences of SEQ ID NOS: 652 to 655, which are partial sequences of ST1C2; and amino acid sequences of SEQ ID NOS: 656 to 659, which are partial sequences of GSTK1, are also the peptides of the present invention. CHST2, MGST3, ST1C2, and GSTK1 can be quantified using any of the corresponding peptides.

Similarly, peptides each consisting of any of amino acid sequences of SEQ ID NOS: 660 to 671, which are partial sequences of CHST7; amino acid sequences of SEQ ID NOS: 672 to 676, which are partial sequences of CHST1; amino acid sequences of SEQ ID NOS: 677 to 683, which are partial sequences of CHST5; amino acid sequences of SEQ ID NOS: 684 to 686, which are partial sequences of NAT6; amino acid sequences of SEQ ID NOS: 687 to 691, which are partial sequences of CHST4; and amino acid sequences of SEQ ID NOS: 692 to 695, which are partial sequences of MAAI, are also the peptides of the present invention. CHST7, CHST1, CHST5, NAT6, CHST4, and MAAI can be quantified using any of the corresponding peptides.

(Peptides Derived from Homologues or Peptides Corresponding to Human SNPs)

Examples of the peptide of the present invention can further include peptides for use in simultaneous protein quantification of metabolizing enzymes with a mass spectrometer, consisting of an amino acid sequence including deletion, substitution, or addition of one or two amino acids in any one of amino acid sequences of SEQ ID NOS: 1 to 695. Specific preferred examples of these peptides can include peptides derived from homologues (homologous proteins) of mammals other than humans or mice, consisting of an amino acid sequence including deletion, substitution, or addition of one or two (preferably one) amino acids in any one of amino acid sequences of SEQ ID NOS: 1 to 695, and peptides corresponding to SNPs in human metabolizing enzyme proteins, consisting of an amino acid sequence including deletion, substitution, or addition of one or two (preferably one) amino acids in any one of partial amino acid sequences of a human metabolizing enzyme protein set forth in SEQ ID NOS: 1 to 413. Examples of the above-mentioned mammals other than human or mouse can include primates other than human (for example, monkey, baboon, and chimpanzee), swine, bovine, equine, goat, sheep, dog, feline, rabbits, guinea pig, gerbil, hamster, and rat.

The above-mentioned peptides consisting of partial amino acid sequences of human metabolizing enzyme proteins, the above-mentioned peptides consisting of partial amino acid sequences of mouse metabolizing enzyme proteins, and the above-mentioned peptides derived from homologues or peptides corresponding to human SNPs (hereinafter, these peptides of the present invention may be collectively referred to as “unlabeled peptide of the invention”) of the present invention can be produced by common chemical synthesis methods. As such a peptide synthesis method, a stepwise elongation method of extending a chain by binding amino acids one by one based on the amino acid sequence information or a fragment condensation method of synthesizing fragments consisting of several amino acids beforehand and then coupling the fragments can be employed.

### (Stable-Isotope-Labeled Peptide)

Furthermore, when the labeled peptide of the present invention is a stable-isotope-labeled peptide in which one or more amino acids constituting the above-mentioned unlabeled peptide of the invention contain any one or more of ¹⁵N, ¹³C, ¹⁸O, and ²H, the type, the position, and the number of the amino acids are not particularly limited so long as the peptides have differences in mass that can be separated by a liquid chromatograph-tandem mass spectrometer (LC-MS/M). Above all, leucine labeled with ¹³C at six sites is more preferably included. Such a stable-isotope-labeled peptide can be chemically synthesized by appropriate measures such as an F-moc method (Amblard M, Fehrentz J A, Martinez J, Subra G. Methods Mol Biol. 298: 3-24 (2005)) using amino acids labeled with a stable isotope. Furthermore, as a stable-isotope-labeled peptide is chemically identical to a peptide to be quantified and shows an identical behavior in LC-MS/MS measurement except that the mass of a labeled amino acid is different, the stable-isotope-labeled peptide can be used as an internal standard peptide in simultaneous protein quantification of metabolizing enzymes with a mass spectrometer.

### (Kit for Quantification and Use as Probes)

The kit of the present invention for simultaneous protein quantification of metabolizing enzymes is not particularly limited so long as the kit comprises the unlabeled peptide of the invention and the stable-isotope-labeled peptide of the present invention. The using method of present invention is not particularly limited so long as the method uses the unlabeled peptide of the invention and the stable-isotope-labeled peptide of the present invention as probes for simultaneous protein quantification of metabolizing enzymes. The unlabeled peptide of the invention, which is also a peptide to be quantified, is used to create a calibration curve for quantification, and the stable-isotope-labeled peptide is used to create a calibration curve for quantification and as an internal standard peptide.

### (Simultaneous Protein Quantification of Metabolizing Enzymes)

The method for simultaneous protein quantification of metabolizing enzymes by LC-MS/MS using the stable-isotope-labeled peptide of the present invention is not particularly limited so long as the method comprises the steps of: (a) performing a mass spectrometry by LC-MS/MS using the unlabeled peptide of the invention and the stable-isotope-labeled peptide of the present invention at predetermined concentration levels to create a calibration curve; (b) performing a mass spectrometry by LC-MS/MS by adding the stable-isotope-labeled peptide of the present invention to peptide fragments obtained by fragmenting metabolizing enzyme proteins to be quantified in a sample with trypsin to calculate a mass spectrum area ratio of a metabolizing enzyme protein peptide to be quantified and a stable-isotope-labeled peptide; and (c) obtaining quantitative values from the area ratio using the calibration curve. Examples of the above-mentioned sample as a source of proteins to be quantified can include various tissues such as the liver and cultured cells derived from various tissues. Furthermore, when the amounts of proteins to be quantified contained in a sample are not more than the measurement limit of a mass spectrometer, a sample fractioned by a method of filling a high pressure nitrogen gas or the like is preferably used in measurement. Furthermore, when two or more peptides to be quantified are identified for one target metabolizing enzyme protein, simultaneous protein quantification of metabolizing enzymes at higher precision and reliability can be achieved by quantification using two or more peptides to be quantified.

Furthermore, when a protein to be quantified and a subtype thereof have very similar amino acid sequences, the protein to be quantified can also be quantified by obtaining a difference between a quantitative value of a peptide common to these proteins and a quantitative value of a peptide specific to one protein. For example, the quantitative value of a metabolizing protein CYP3A4 can be obtained by (a quantitative value of a peptide common to CYP3A4 and CYP3A5)—(a quantitative value of a peptide specific to CYP3A5).

Hereafter, the present invention will be specifically described with reference to the following Examples. However, the technical scope of the present invention is not limited to these examples.

### Example 1

### Detection of Peptides (CYP and P450R) by nanoLC-MS/MS

Measurement by nanoLC-MS/MS was performed using peptides to be quantified, which are partial sequence peptides of human metabolizing enzymes CYP and P450R (unlabeled peptides: CYP1A2, YLPNPALQR (SEQ ID NO: 7); CYP2A6, GTGGANIDPTFFLSR (SEQ ID NO: 15); CYP2B6, GYGVIFANGNR (SEQ ID NO: 25); CYP2C8, GNSPISQR (SEQ ID NO: 28); CYP2C9, GIFPLAER (SEQ ID NO: 30); CYP2C18, IAENFAYIK (SEQ ID NO: 32); CYP2C19, GHFPLAER (SEQ ID NO: 35); CYP2D6, DIEVQGFR (SEQ ID NO: 39); CYP2E1, GIIFNNGPTWK (SEQ ID NO: 44); CYP2J2, EENGQPFDPHFK (SEQ ID NO: 52); CYP3A4, LQEEIDAVLPNK (SEQ ID NO: 81); CYP3A5, DTINFLSK (SEQ ID NO: 83); CYP3A7, FNPLDPFVLSIK (SEQ ID NO: 85); CYP3A43, YIPFGAGPR (SEQ ID NO: 91); CYP4A11, IPIPIAR (SEQ ID NO: 100); CYP51A1, FAYVPFGAGR (SEQ ID NO: 290); and P450R, FAVFGLGNK (SEQ ID NO: 355)) and synthesized stable-isotope-labeled peptides [isotope-labeled ¹³C₆, peptides: CYP1A2, YLPNPAL (¹³C₆, (SEQ ID NO: 7); CYP2A6, GTGGANIDPTFFL (¹³C₆,¹⁵N) SR (SEQ ID NO: 15); CYP2B6, GYGVIFA(¹³C₆,¹⁵N)NGNR (SEQ ID NO: 25); CYP2C8, GNSPI (¹³C₆,¹⁵N) SQR (SEQ ID NO: 28); CYP2C9, GIFPL(¹³C₆,¹⁵N)AER (SEQ ID NO: 30); CYP2C18, IAENFAYI (¹³C₆,¹⁵N)K (SEQ ID NO: 32); CYP2C19, GHFPL(¹³C₆,¹⁵N) AER(SEQ ID NO: 35); CYP2D6, DIEVQGF (¹³C₆,¹⁵N)R (SEQ ID NO: 39); CYP2E1, GIIFNNGP(¹³C₆,¹⁵N)TWK (SEQ ID NO: 44); CYP2J2, EENGQPFDPHF(¹³X₆,¹⁵N)K (SEQ ID NO: 52); CYP3A4, LQEEIDAVLP(¹³C₆, ^{‥}N)NK (SEQ ID NO: 81); CYP3A5, DTINFL (¹³C₆,¹⁵N)SK (SEQ ID NO: 83); CYP3A7, FNPLDPFVLSI (¹³C₆,¹⁵N)K (SEQ ID NO: 85); CYP3A43, YIPFGAGP(¹³C₆,¹⁵N)R (SEQ ID NO: 91); CYP4A11, IPIPIA(¹³C₆, ¹⁻⁵N) R (SEQ ID NO: 100); CYP51A1, FAYVPFGA (¹³C₆,¹⁵N)GR (SEQ ID NO: 290); and P450R, FAVFGL(¹³C₆,¹⁵N)GNK (SEQ ID NO: 355)] under the following conditions.

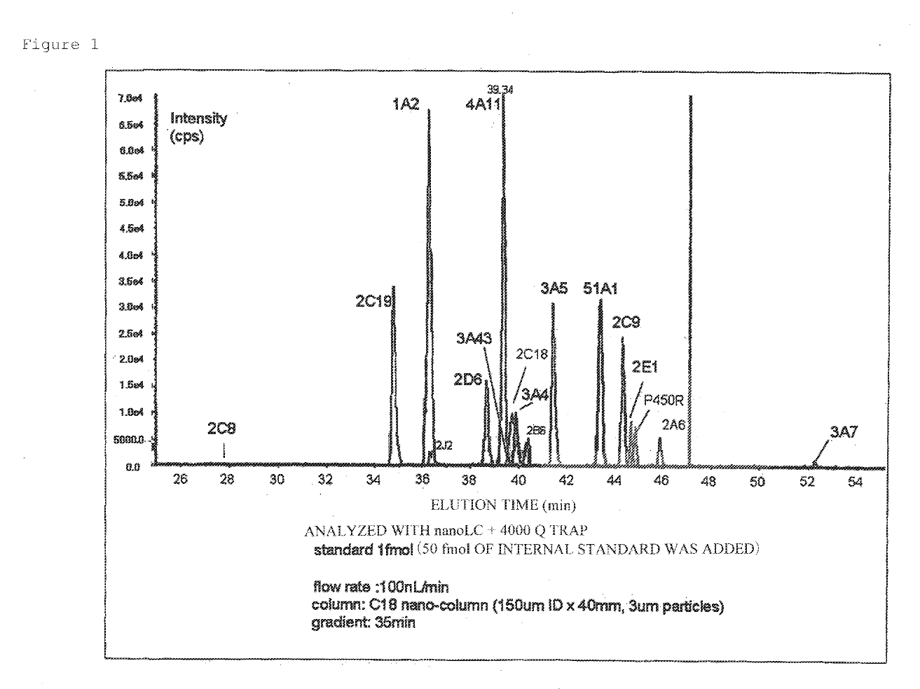

50 fmol each of ¹³C₆,¹⁵N-labeled peptides were added to 1 fmol of unlabeled peptides for measurement by nanoLC-MS/MS. A reverse phase C18 150 μm I.D.×40 mm was used as a column. 0.1% formic acid and 0.1% formic acid/acetonitrile were used as mobile phases. Analysis was performed with a linear gradient of increasing the concentration of 0.1% formic acid/acetonitrile to 45% over 35 minutes. 4000 Q Trap of Applied Biosystems was used as amass spectrometer. A peptide was identified when a peptide to be quantified and a labeled peptide were eluted with the same time. The results are shown in FIG. 1.

### Example 2

### Simultaneous Quantification of CYP and P450R in Human Liver Tissue Sample

A human liver tissue was shredded with scissors and then suspended in 10 mM. Tris-HCl, 10 mM NaCl, and 1.5 mM MgCl₂. The suspension was homogenized with a glass homogenizer, and the solution was centrifuged at 8000×g for 10 minutes. Further, the supernatant was centrifuged at 100,000×g for 60 minutes to obtain a crude membrane fraction. The obtained crude membrane fraction was denatured in a buffer containing 7 M guanidine hydrochloride, 0.1 M Tris-HCl, and 10 mM EDTA (pH 8.5) and subjected to reduction with DTT and carbamide methylation with iodoacetamide to protect the SH group of a cysteine residue. Concentration and desalting were performed by a methanol chloroform precipitation method. The fraction was suspended in 1.2 M urea and 10 mM Tris-HCl, then trypsin was added in an amount corresponding to 1/100 of the protein weight, and proteins were digested with the enzyme at 37° C. for 16 hours to obtain a peptide sample.

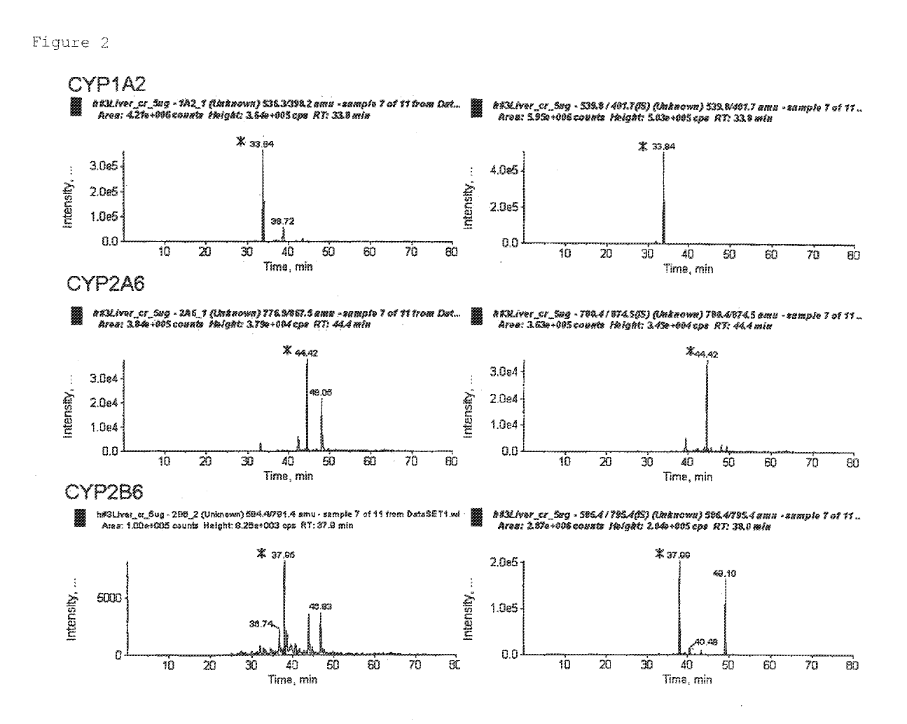

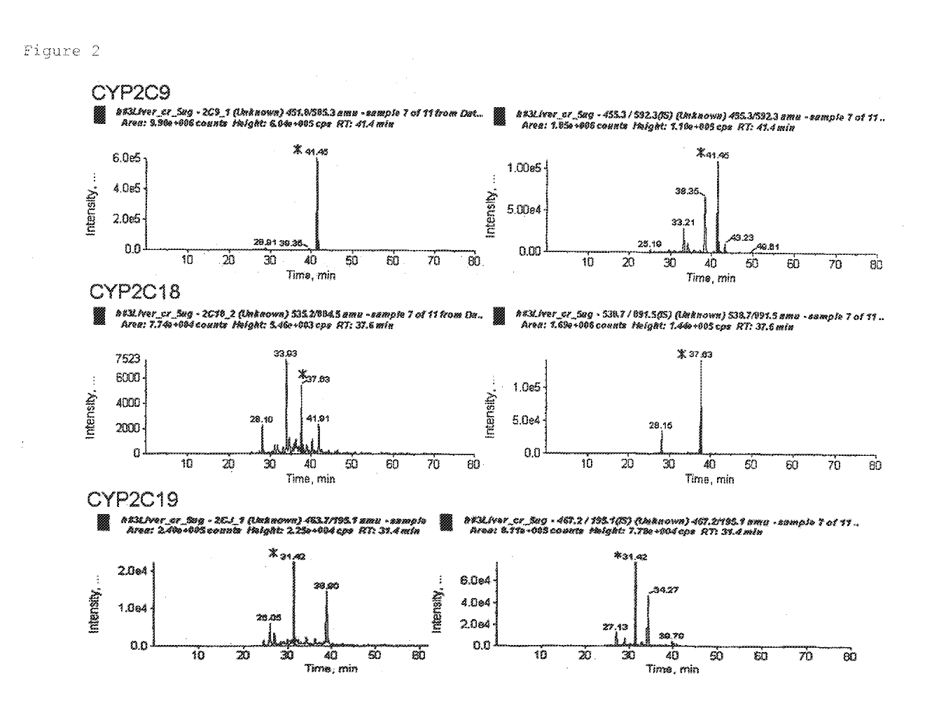

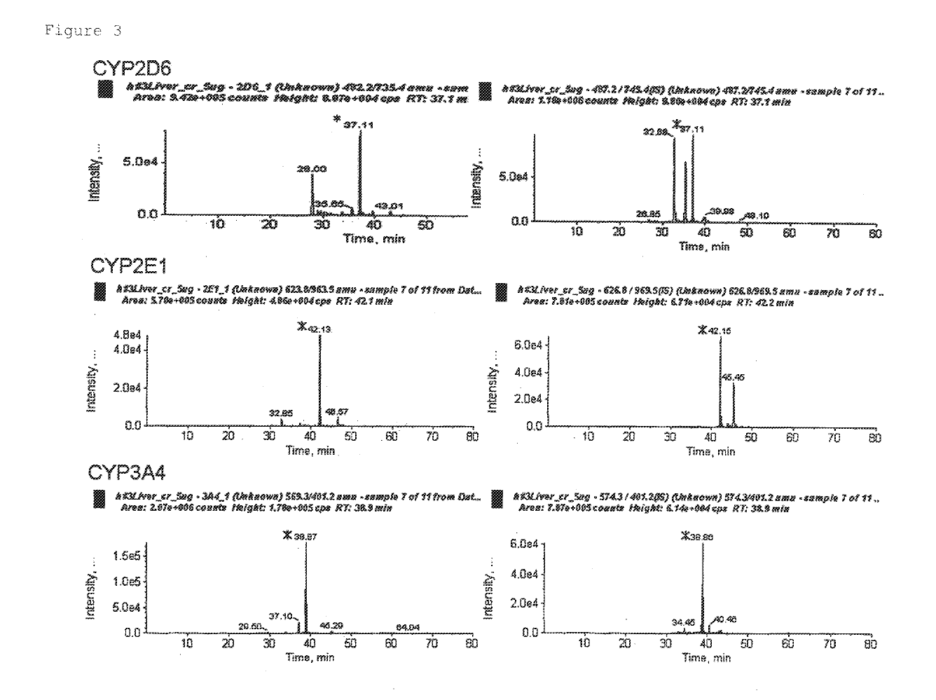

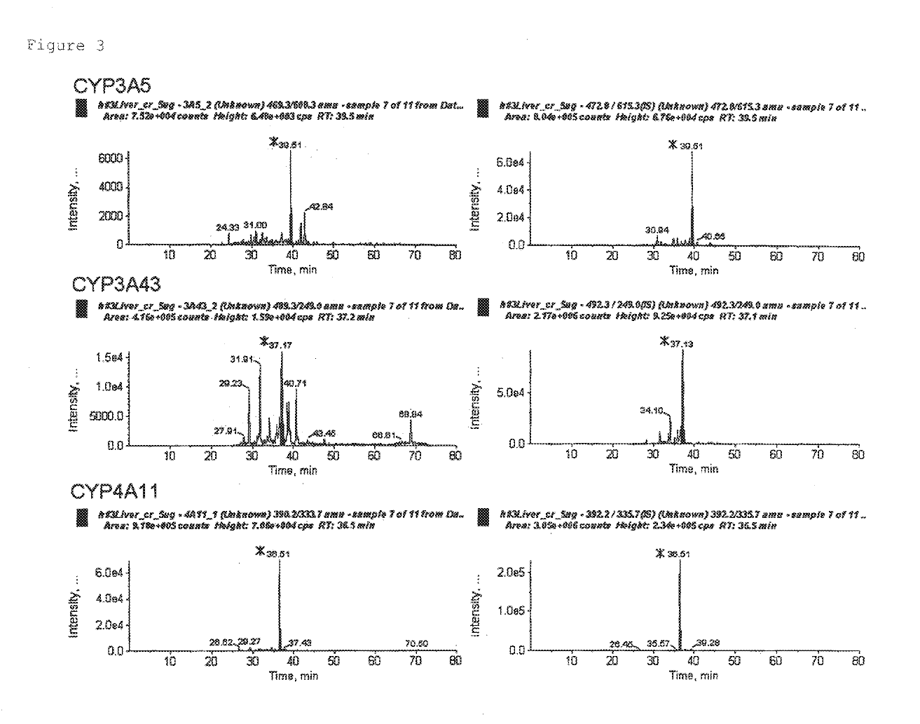

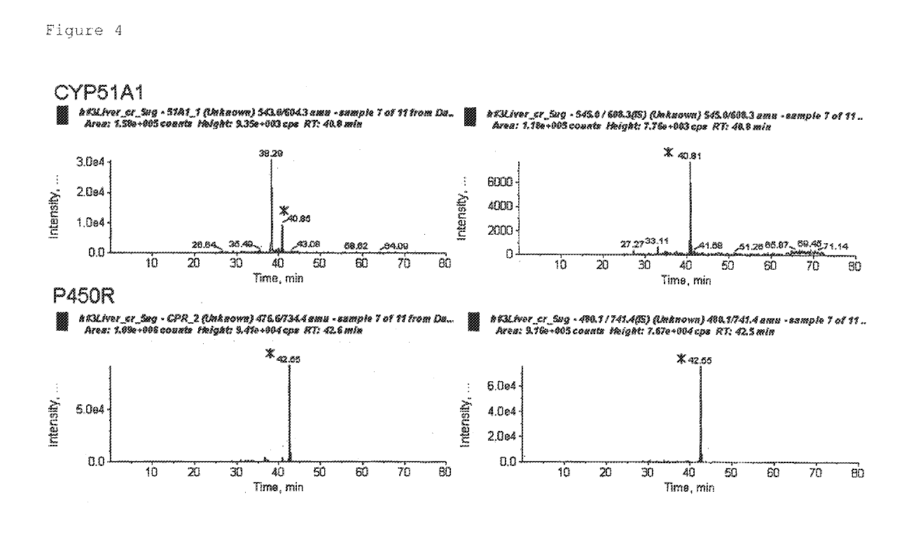

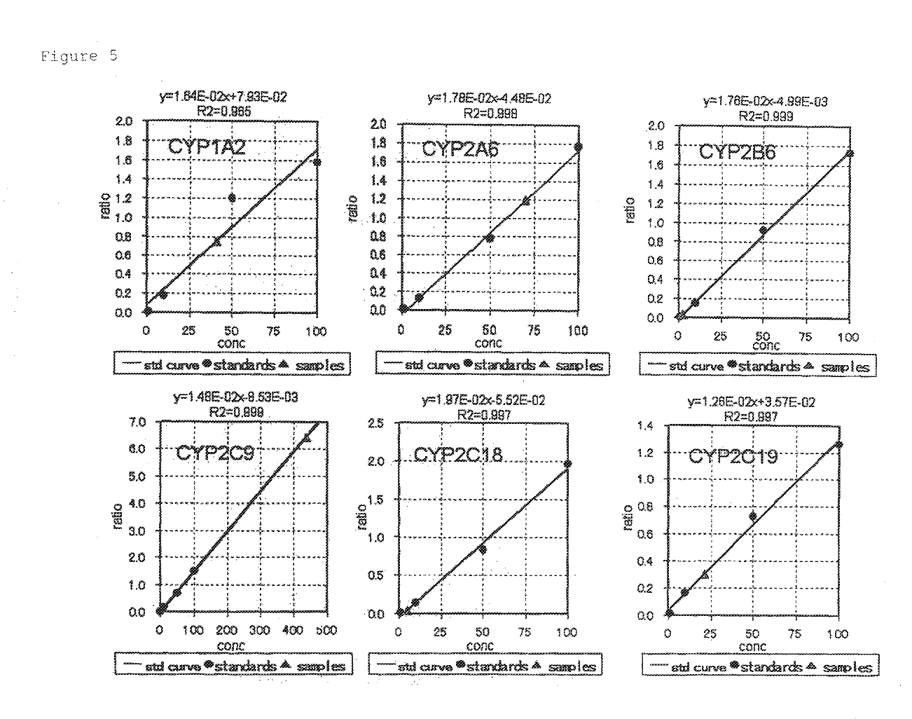

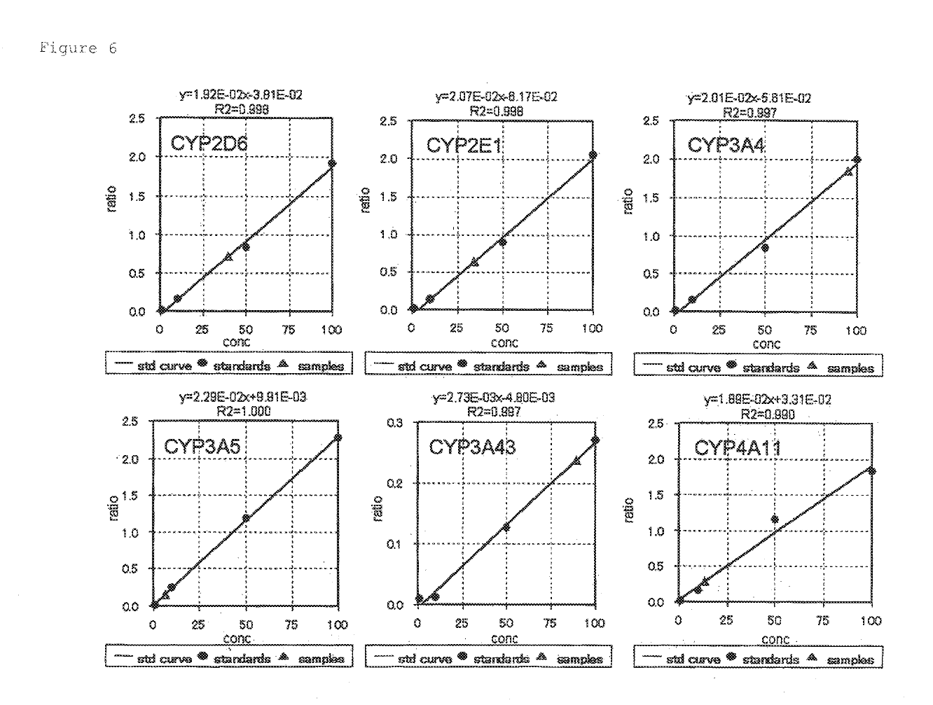

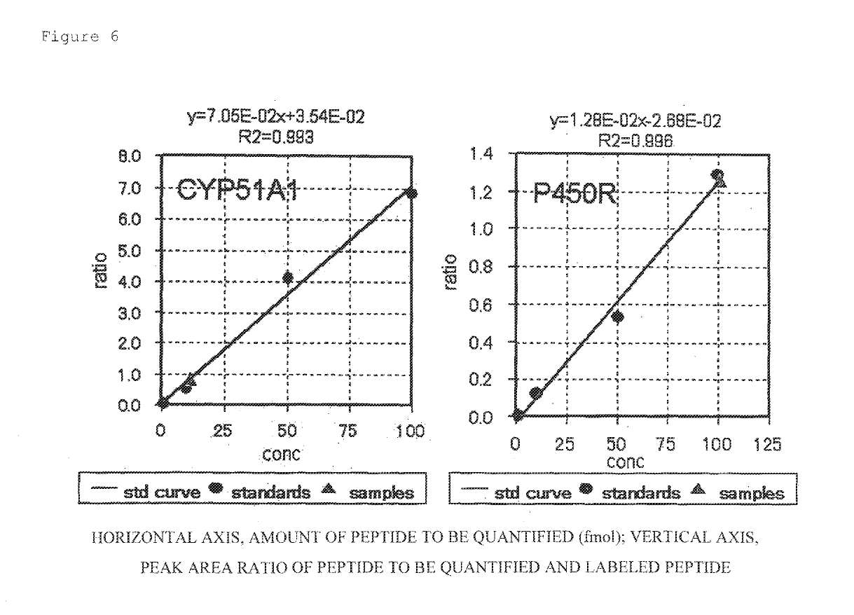

50 fmol of ¹³C₆,¹⁵N-labeled peptides were added to 5 μg of the obtained crude membrane fraction peptide sample, and peptides were measured by LC-MS/MS (FIG. 2). After the measurement, MS spectrum area ratios (unlabeled peptide/¹³C₆,¹⁵N-labeled peptide) were calculated, and quantitative values were obtained using a calibration curve.

### (Creation of Calibration Curve)

A calibration curve was created using a selected peptide to be quantified to examine linearity. 50 fmol each of ¹³C₆,¹⁵N-labeled peptides were added to each of 1, 5, 10, 50, and 100 fmol of an unlabeled peptide, and peptides were similarly measured by nanoLC-MS/MS. MS spectrum area ratios (unlabeled peptide/¹³C₆,¹⁵N-labeled peptide) were calculated to create a calibration curve.

The quantification results CYP and P450R are shown in Table 1.

TABLE 1

| Metabolizing enzyme | Quantitative value |
|---|---|
| CYPorP450R | (fmol/assay) |
| CYP1A2 | 40.8 |
| CYP2A6 | 70.0 |
| CYP2B6 | 2.50 |
| CYP2C9 | 435 |
| CYP2C18 | 5.00 |
| CYP2C19 | 21.1 |
| CYP2D6 | 39.1 |
| CYP2E1 | 33.9 |
| CYP3A4 | 95.1 |
| CYP3A5 | 6.50 |
| CYP3A43 | 89.0 |
| CYP4A11 | 13.5 |
| CYP51A1 | 11.3 |
| P450R | 100 |

### Example 3

### Detection of Peptides (Ara-C Metabolizing Enzymes) by Conventional LC-MS/MS

Measurement by conventional LC-MS/MS was performed using peptides to be quantified, which are partial sequence peptides of human Ara-C metabolizing enzymes, (unlabeled peptides: dCK, AQLASLNGK (SEQ ID NO: 362); CDA, AVSEGYK (SEQ ID NO: 364); cN-IA, GFLEALGR (SEQ ID NO: 366); cN-IB, GFLEDLGR (SEQ ID NO: 369); cN-II, VFLATNSDYK (SEQ ID NO: 375); cN-III, GELIHVFNK (SEQ ID NO: 379); dNT-1, TVVLGDLLIDDK (SEQ ID NO: 383); Ecto-5′-NT, VPSYDPLK (SEQ ID NO: 388); RRM1, LNSAIIYDR (SEQ ID NO: 390); RRM2, ENTPPALSGTR (SEQ ID NO: 393); UMP/CMPK, FLIDGFPR (SEQ ID NO: 395); dCMPDA, LIIQAGIK (SEQ ID NO: 396); CTPS1, FVGQDVEGER (SEQ ID NO: 402); CTPS2, ADGILVPGGFGIR (SEQ ID NO: 409)) and synthesized stable-isotope-labeled peptides [isotope-labeled peptides: dCK, AQLASL(¹³C₆,¹⁵N)NGK (SEQ ID NO: 362); CDA, AV (¹³C₆,¹⁵N)SEGYK (SEQ ID NO: 364); cN-IA, GFLEAL (¹³C₆,¹⁵N)GR (SEQ ID NO: 366); cN-IB, GFLEDL (¹³C₆,¹⁵N) GR (SEQ ID NO: 369); cN-II, VFLA(¹³C₆,¹⁵N) TNSDYK (SEQ ID NO: 375); cN-III, GELIHVF(¹³C₆,¹⁵N) NK(SEQ ID NO: 379); dNT-1, TVVLGDLLI (¹³C₆,¹⁵N)DDK (SEQ ID NO: 383); Ecto-5′-NT, VPSYDPL(¹³C₆,¹⁵N)K (SEQ ID NO: 388); RRM1, LNSAII (¹³C₆, ¹⁵N) YDR (SEQ ID NO: 390); RRM2, ENTPPAL (¹³C₆,¹⁵N)SGTR (SEQ ID NO: 393); UMP/CMPK, FLIDGFP (¹³C₆,¹⁵N)R (SEQ ID NO: 395); dCMPDA, LIIQAGI (¹³C₆,¹⁵N)K (SEQ ID NO: 396); CTPS1, FVGQDV(¹³C₆, ⁵N)EGER (SEQ ID NO: 402); and CTPS2, ADGILVPGGFGI (¹³C₆,¹⁵N)R (SEQ ID NO: 409)] under the following conditions.

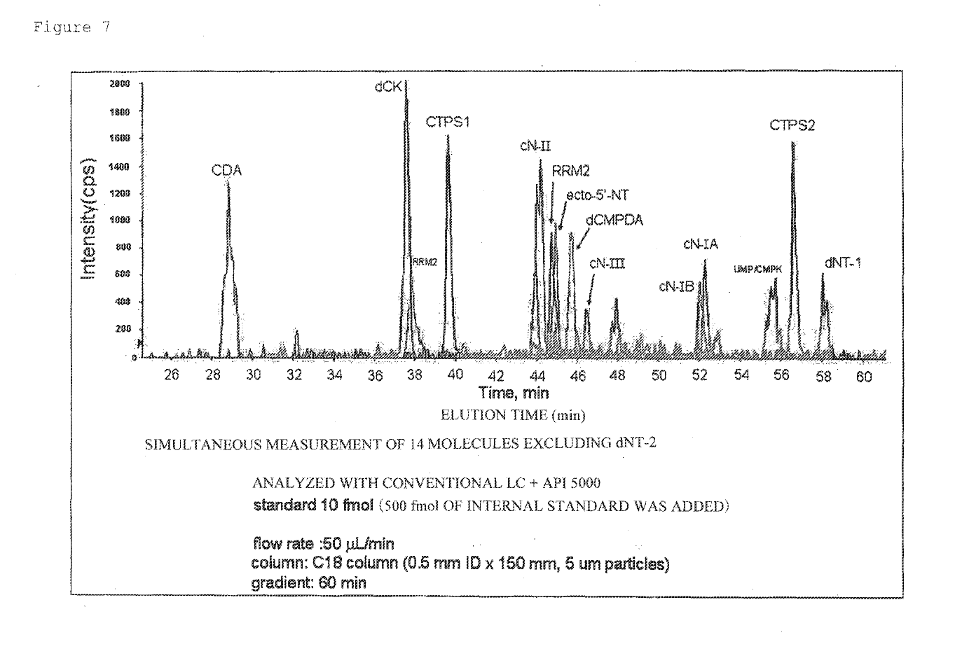

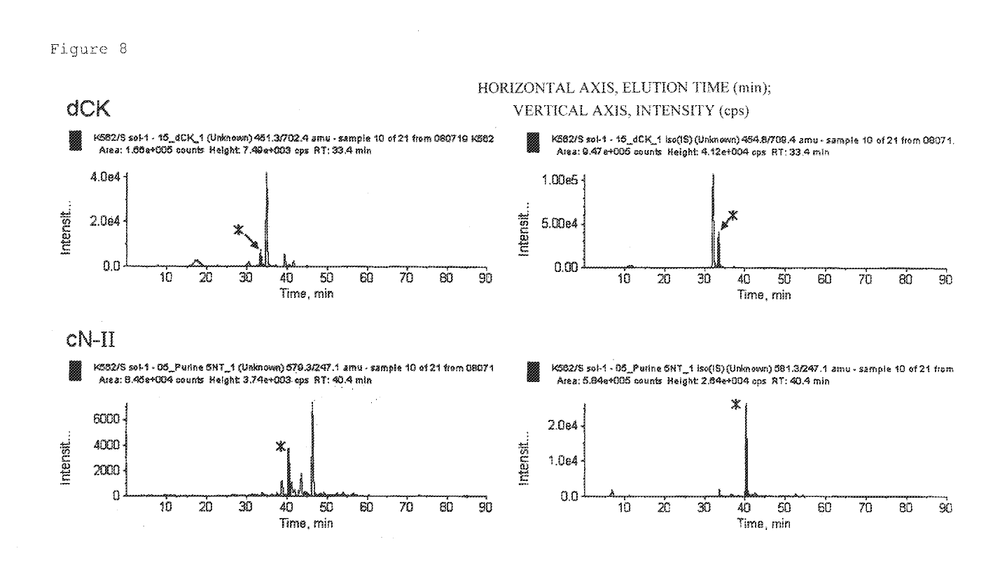

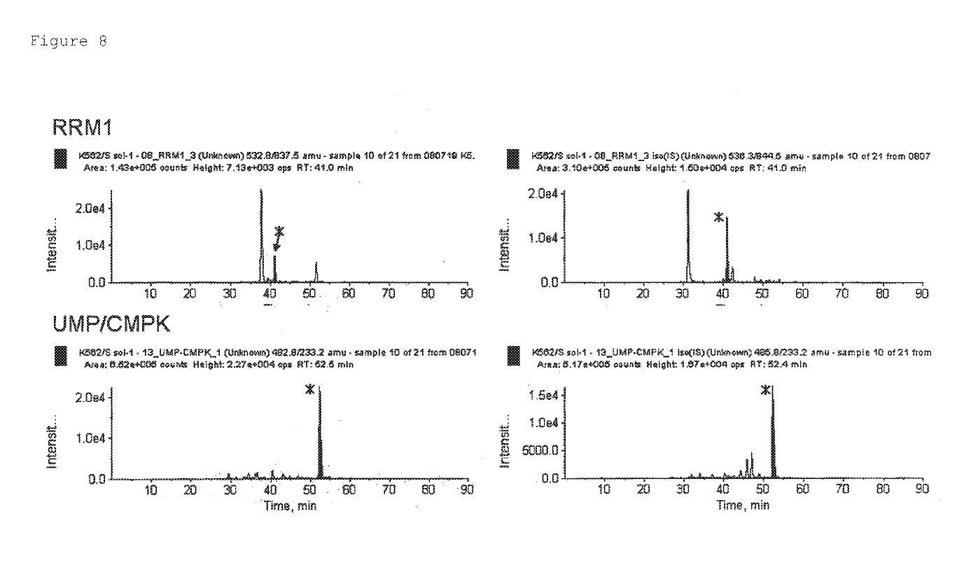

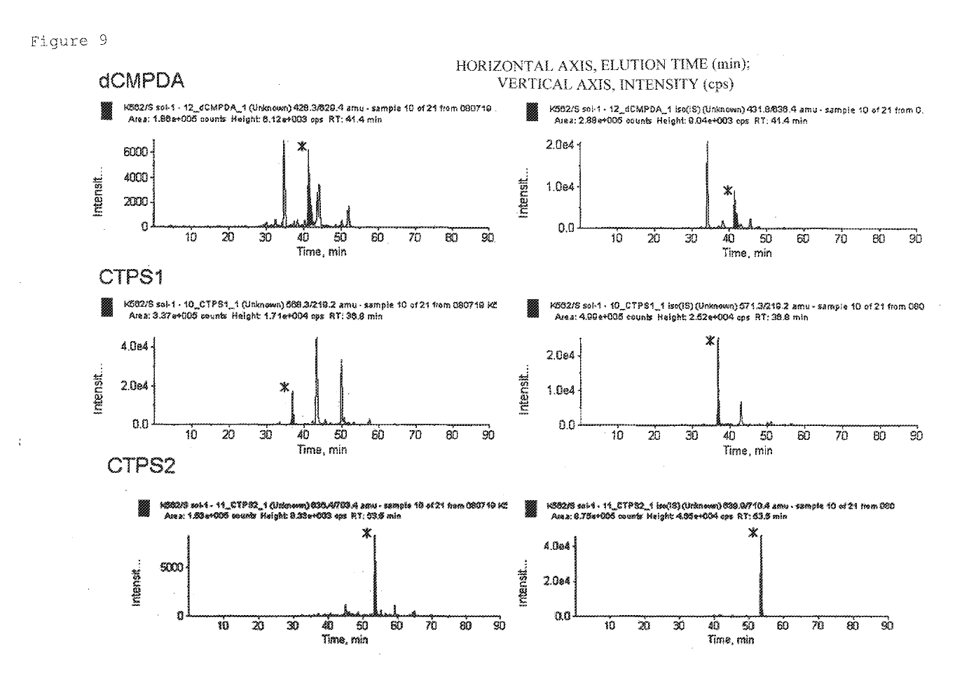

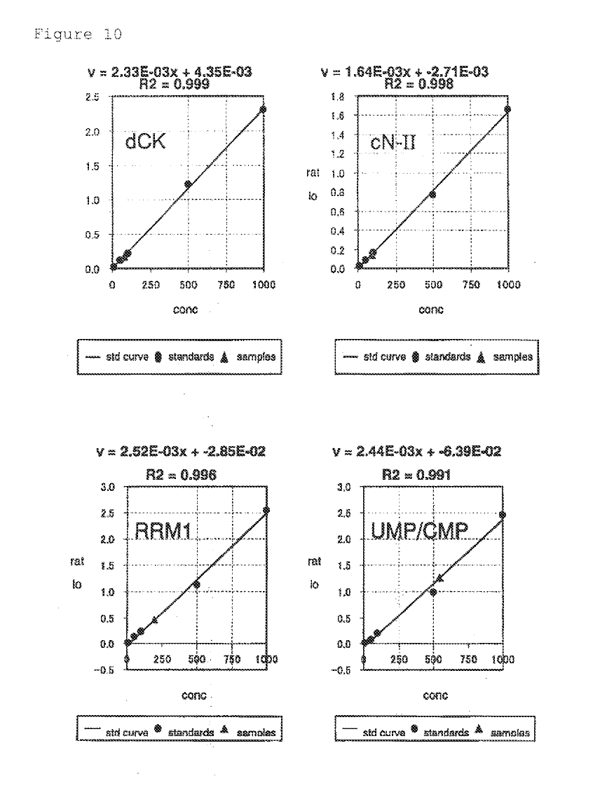

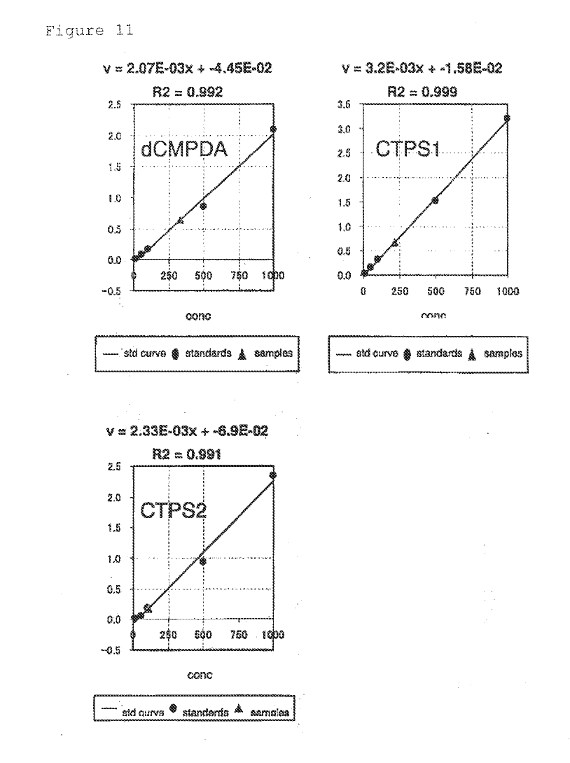

500 fmol each of ¹³C₆,¹⁵N-labeled peptides were added to 10 fmol of unlabeled peptides for measurement by conventional LC-MS/MS. A reverse phase C18 0.5 mm I.D.×150 mm was used as a column. 0.1% formic acid and 0.1% formic acid/acetonitrile were used as mobile phases. Analysis was performed with a linear gradient of increasing the concentration of 0.1% formic acid/acetonitrile to 45% over 60 minutes. API 5000 of Applied Biosystems was used as a mass spectrometer. A peptide was identified when a peptide to be quantified and a labeled peptide are eluted with the same time. The results are shown in FIG. 7.

### Example 4

### Simultaneous Quantification of Ara-C Metabolizing Proteins in Human Cultured Cells

5.0×10⁸ cells of human leukemic cell strain K562 were cultured in an RPMI-1640 medium containing 10% FBS and then crushed by a cavitation method. This solution was centrifuged at 10,000×g for 10 minutes. Then, the supernatant was centrifuged at 100,000×g for 60 minutes to obtain a soluble fraction. The obtained soluble fraction was denatured in a buffer containing 7 M guanidine hydrochloride, 0.1 M Tris-HCl, and 10 mM EDTA (pH 8.5) and subjected to reduction with DTT and carbamide methylation with iodoacetamide to protect the SH group of a cysteine residue. Concentration and desalting were performed by a methanol chloroform precipitation method. The fraction was suspended in 1.2 M urea and 10 mM Tris-HCl, then trypsin was added in an amount of 1/100 corresponding to the protein weight, and proteins were digested with the enzyme at 37° C. for 16 hours to obtain a peptide sample.

500 fmol of ¹³C₆,¹⁵N-labeled peptides were added to 50 μg of the obtained crude membrane fraction peptide sample and measured by LC-MS/MS. After measurement, MS spectrum area ratios (¹³C₆,¹⁵N-labeled peptide) were calculated, and quantitative values were obtained using the calibration curve.

### (Creation of Calibration Curve)

A calibration curve was created using a selected peptide to be quantified. 500 fmol each of ¹³C₆,¹⁵N-labeled peptides were added to 1, 5, 10, 50, 100, 500, and 1000 fmol of an unlabeled peptide and measured by conventional LC-MS/MS in the same manner as in Example 1. The MS spectrum area ratios (unlabeled peptide/¹³C₆,¹⁵N-labeled peptide)were calculated to create a calibration curve.

The results of Ara-C metabolizing enzyme quantification are shown in Table 2.

TABLE 2

| Ara-C-related | Quantitative value |
|---|---|
| metabolizing enzyme | (fmol/assay) |
| dCK | 73.5 |
| cN-II | 89.9 |
| RRM1 | 195 |
| UMP/CMPK | 544 |
| dCMPDA | 334 |
| CTPS1 | 216 |
| CTPS2 | 104 |

### Comparative Example 1

LC-MS/MS measurement was performed under the following conditions using a peptide to be quantified (unlabeled peptide: ENT2, VALTLDLDLEK (SEQIDNO: 696)), which is apartial sequence peptide of a human transporter ENT2 (SLC29A2), and a synthesized stable-isotope-labeled peptide [isotope-labeled peptide: ENT2, VALTLDLDL (¹³C₆,¹⁵N)EK (SEQ ID NO: 696)], but the peptide could not be quantified due to the low detection sensitivity.

500 fmol of the ¹³C₆,¹⁵N-labeled peptide was added to 500 fmol of the unlabeled peptide and measured by conventional LC-MS/MS. A reverse phase C18 0.5 mm I.D.×150 mm was used as a column. 0.1% formic acid and 0.1% formic acid/acetonitrile were used as mobile phases. Analysis was performed with a linear gradient of increasing the concentration of 0.1% formic acid/acetonitrile to 45% over 60 minutes. API 5000 of Applied Biosystems was used as a mass spectrometer. The peptide was identified under a condition that the peptide to be quantified and the labeled peptide were eluted with the same time, but no peaks were detected for either the peptide to be quantified or the labeled peptide due to the low sensitivity.

### Comparative Example 2

LC-MS/MS measurement was performed under the following conditions using a peptide to be quantified (unlabeled peptide: BGT1, QELIAWEK (SEQ ID NO: 697)), which is a partial sequence peptide of a human transporter BGT1 (SLC6A12), and a synthesized stable-isotope-labeled peptide [isotope-labeled peptide: BGT1, QELIA(¹³C₆,¹⁵N)WEK (SEQ ID NO: 697)], but the peptide could not be quantified because peptides were not retained on the reverse phase column and therefore the sensitivity was low.

500 fmol of the ¹³C₆,¹⁵N-labeled peptide was added to 500 fmol of the unlabeled peptide and measured by conventional LC-MS/MS. A reverse phase C18 0.5 mm I.D.×150 mm was used as a column. 0.1% formic acid and 0.1% formic acid/acetonitrile were used as mobile phases. Analysis was performed with a linear gradient of increasing the concentration of 0.1% formic acid/acetonitrile to 45% over 60 minutes. API 5000 of Applied Biosystems was used as a mass spectrometer. The peptide was identified under a condition that the peptide to be quantified and the labeled peptide were eluted with the same time, but no peaks were detected for either the peptide to be quantified or the labeled peptide.

1. A peptide for use in simultaneous protein quantification of metabolizing enzymes with a mass spectrometer, consisting of the partial amino acid sequence of a human metabolizing enzyme protein set forth in any one of SEQ ID NOS: 1 to 412.

2. A peptide for use in simultaneous protein quantification of metabolizing enzymes with a mass spectrometer, consisting of the partial amino acid sequence of a mouse metabolizing enzyme protein set forth in any one of SEQ ID NOS: 413 to 695.

3. A peptide for use in simultaneous protein quantification of metabolizing enzymes with a mass spectrometer, consisting of an amino acid sequence including deletion, substitution or addition of one or two amino acids in the amino acid sequence set forth in any one of SEQ ID NOS: 1 to 695.

4. A stable-isotope-labeled peptide wherein one or more of peptide-constituting amino acids contain any one or more of ¹⁵N, ¹³C, ¹⁸O, and ²H in the peptide according to any one of claims 1 to 3.

5. A kit for simultaneous protein quantification of metabolizing enzymes, comprising the peptide according to any one of claims 1 to 3 and the stable-isotope-labeled peptide according to claim 4.

6. A method of using the peptide according to any one of claims 1 to 3 and the stable-isotope-labeled peptide according to claim 4 as a probe for simultaneous protein quantification of metabolizing enzymes.

7. A method for simultaneous protein quantification of metabolizing enzymes with a liquid chromatograph-tandem mass spectrometer (LC-MS/MS) using a stable-isotope-labeled peptide, comprising the following steps (a) to (c):
(a) performing a mass spectrometry by LC-MS/MS using the peptide according to any one of claims 1 to 3 and the stable-isotope-labeled peptide according to claim 4 at predetermined concentration levels to create a calibration curve;
(b) performing a mass spectrometry by LC-MS/MS by adding the stable-isotope-labeled peptide according to claim 4 to a peptide fragment obtained by fragmenting metabolizing enzyme proteins to be quantified in a sample with trypsin to calculate a mass spectrum area ratio of a metabolizing enzyme protein peptide to be quantified and a stable-isotope-labeled peptide; and
(c) obtaining a quantitative value from the area ratio using a calibration curve.
